(19) United States
(12) Patent Application Publication
(10) Pub. No.: US 2005/0009060 A1
(43) Pub. Date: Jan. 13, 2005

(54) Multiplexed multitarget screening method

(76) Inventors: Andrew Beernink, San Diego, CA (US); Teresa A. Bennett, San Diego, CA (US); Alex Okun, San Diego, CA (US); Juan A. Ballesteros, San Diego, CA (US); John T. Ransom, Encinitas, CA (US)

Correspondence Address: PILLSBURY WINTHROP LLP, ATTENTION: DOCKETING DEPARTMENT, 11682 EL CAMINO REAL, SUITE 200, SAN DIEGO, CA, 92130, US

(21) Appl. No.: 10/841,410

(22) Filed: May 7, 2004

Related U.S. Application Data
(60) Provisional application No. 60/469,089, filed on May 7, 2003.

(51) Int. Cl. C12Q 1/68; G01N 1/30; G01N 33/48

(52) U.S. Cl. 435/6; 435/40.5

(57) ABSTRACT

The present disclosure provides systems for multiplexed multitarget screening of cell populations having one or more wild type or mutated ligand targets and measuring cell responses to ligands using high throughput screening techniques, including flow cytometry (FCM). The method includes the steps of: 1) developing cell populations to be screened; 2) staining cell populations using one or more fluorochromes to yield a distinct excitation/emission signature for each cell population; 3) combining labelled cell populations into a single mixed suspension; 4) analyzing populations to resolve them on the basis of their unique signature; and 5) resolving individual populations and deconvoluting data to extract meaningful information about populations.

### RELATED APPLICATIONS

This application claims benefit of priority to U.S. Provisional Patent Application Ser. No. 60/469,089, filed May 7, 2003.

### FIELD OF THE INVENTION

The present invention relates generally to screening multiple populations having one or more wild type or variant ligand targets. More specifically, the present invention relates to the measurement of responses to test candidate drug compounds using high throughput screening techniques, including flow cytometry.

### BACKGROUND OF THE INVENTION

Flow cytometry is a technique that can provide multiparametric data about the physical and chemical characteristics of cells in suspension. The flow cytometer has an hydraulic (or fluidic) system, an optical system, and electronics system and a computer system. The hydraulic system is composed of a group of pneumatic controls and fluidic delivery systems to establish a laminar flow through hydrodynamic focusing that allows cells in the cellular suspension to move in single file through the flow chamber. The optical system consists of a light source, filters, lenses and mirrors as necessary, and detectors. The light source is usually produced by a laser or laser diode. Commonly, multiple laser light sources are used, thereby increasing the flexibility and precision of the flow cytometric analysis. One commonly used light source is a gas laser (commonly argon), usually cooled by circulating air or water, that produces a monochrome light of 488 nm. Light produced by the light source(s) is used to excite fluorochrome labels attached to cells, which results in the emission of light by the fluorochrome with a characteristic spectral profile. The fluorescence emissions of one or more fluorochromes are typically selected to yield optimal and resolvable emission signatures, often by steering dichroic filters and bandpass filters so that the emissions signals from individual fluorochromes are directed to distinct photodetectors. This allows the fluorescent signal distribution for each fluorochrome to be individually detected and resolved from others present on the cell. The electronics system is used for signal detection, e.g., fluorescent emissions and scattered excitation light signals, data processing, and automation. Finally, the computer system receives digitized information from the cytometer and processes it for later analysis. Integration of these systems allows accurate and fast analysis of a high number of cells in little time.

Flow cytometric measurements can be made on several different characteristics of each cell. The fact that flow cytometric analysis can be carried out on individual cells in a mixture allows more accurate analysis because the responses of individual cells are measured, while standard screening systems, e.g., fluorometric imaging plate readers (FLIPR™), measure only the average response of a population of cells. Multiparametric measurements of individual cells in a mixture allow one to correlate different characteristics of cells and thus define subpopulations and/or distinguish between different cell types. Typical commercial flow cytometers allow 5-20 different parameters to be collected for each cell, allowing a multidimensional representation of a population to be obtained.

### SUMMARY OF THE INVENTION

The present disclosure provides a multiplexed screening method including the steps of: (a) developing a plurality of cell populations to be screened, wherein each cell population expresses a ligand target; (b) color-coding each of the cell populations by staining at least one of the cell populations with a fluorochrome, to yield a distinct optical signature for each color-coded cell population, and loading each cell population with a fluorescent indicator dye to monitor a cellular response; (c) combining the color-coded cell populations to form a mixed cell suspension; (d) contacting the mixed cell suspension with a ligand or control compound; (e) analyzing the mixed cell suspension by a single cell analysis system, using one or more light sources to excite each color-coded cell in the mixed cell suspension and collecting fluorescence emissions from each excited cell to measure the distinct optical signature and the cellular response of each cell; and (f) resolving each of the cell populations in the mixed cell suspension by deconvoluting data collected in step (e).

For color-coding, cells can be stained with one or more flurochromes including, but not limited to, FM 1-43, FM 4-64, DiO, DiI, DiA, DiD, DiR, PKH 2, PKH26, Bodipy 665, LysoSensor Blue, Hoescht 33232, fluorescein, coumarin, rhodamine. Alexa 488, Alexa 500, Alexa 514, phycoerythrin, Alexa 594, Alexa 647, Alexa 660 and Alexa 750. The cell populations can be directly stained with at least one fluorochrome, or can be indirectly stained using a two-step process in which the cell populations are first labelled with an anchor molecule and then at least one fluorochrome binds to the anchor molecule. In the indirect staining method, the anchor molecule can be a biotinylated molecule and the fluorochrome is conjugated to a biotin-binding molecule such as streptavidin or avidin. Likewise, the anchor molecule can be an avidin- or streptavidin-conjugated molecule and the fluorochrome is conjugated to an avidin- or streptavidin-binding molecule such as biotin or a biotin derivative. A cell population can also be color-coded by not staining with a flurochorome, which also yields a distinct optical signature for the unstained cell population.

In the multiplexed screening method provided herein, the cellular response that is measured with the indicator dye is a cellular response to the ligand. The cellular response to the ligand can be Ca²⁺ mobilization (Ca²⁺ᵢ), which can be measured Indo-1, Fluo-3, Fluo-4, Oregon Green 488 BAPTA, Calcium Green, X-rhod-1 or Fura Red as the indicator dye. The cellular response to the ligand can be a change in membrane potential.

The mixed cell suspension can be incubated with the ligand for any suitable period before analyzing, including from between about 0.1 second to about 1 week, between about 1 second to about 5 seconds, between about 1 minute to about 1 hour, or between about 1 hour to about 48 hours. Additional indicator dyes can be added to the mixture before analyzing.

In the multiplexed screening method provided herein, the plurality of cell populations can include one or more of any of the following, alone or in combination: a cell population expressing an endogenous ligand target; a cell population expressing a transfected ligand target; a cell population expressing a regulatable ligand target; a cell population expressing a ligand target with an expression tag; a cell population expressing a wild type ligand target; a cell population expressing a variant ligand target, where the variant ligand target can be a naturally occurring variant ligand target or a mutant ligand target. The plurality of cell populations can include a cell population expressing a wild type ligand target and at least one cell population expressing a variant ligand target. The plurality of cell populations can include a plurality of cell populations expressing distinct variant ligand targets.

In the multiplexed screening method provided herein, analyzing the mixed cell suspension and resolving each of the plurality of cell populations in the mixed cell suspension can be used to identify cell populations having increased response to the ligand. Cells having increased response to the ligand are isolated.

In the multiplexed screening method provided herein, the single cell analysis system can be a flow cytometry system, in particular a flow cytometry system with fluorescence activated cell sorting (FACS). Cells can be sorted and isolated using FACS.

In the multiplexed screening method provided herein, the single cell analysis system can include a liquid handling apparatus that is operative to prepare a mixed cell suspension, a sample analysis apparatus, and an injection guide coupled to the analysis apparatus, wherein the injection guide is operative to receive the mixed cell suspension from the liquid handling apparatus and provide the mixed cell suspension to a fluidic system of the sample analysis apparatus.

### BRIEF DESCRIPTION OF THE DRAWINGS

FIG. 1 is a simplified block diagram illustrating functional components of one embodiment of a sample analysis system incorporating elements of a direct sample injection system.

FIG. 2 is a simplified block diagram illustrating functional components of another embodiment of a sample analysis system incorporating elements of a direct sample injection system.

FIG. 3 is a simplified flow diagram illustrating the general operation of one embodiment of a method of performing an analysis using a direct sample injection system.

FIG. 4 is a simplified flow diagram illustrating the general operation of another embodiment of a method of performing an analysis using a direct sample injection system.

FIG. 5 is a simplified diagram illustrating a perspective view of one embodiment of a sample injection guide engaged with a pipette tip during use.

FIG. 6 is a simplified diagram illustrating a perspective view of one embodiment of a coupling component allowing a pipette probe to engage a pipette tip.

FIG. 7 is a simplified diagram illustrating a side elevation view of the coupling component embodiment of FIG. 6.

FIG. 8 is a simplified diagram illustrating an axial view of the coupling component embodiment of FIG. 6.

FIG. 9 is a simplified diagram illustrating a perspective view of one embodiment of a sample injection guide.

FIG. 10 is a simplified diagram illustrating a plan view of the sample injection guide embodiment of FIG. 9.

FIG. 11 is a simplified diagram illustrating a side elevation view of the sample injection guide embodiment of FIG. 9.

FIG. 12 is a simplified diagram illustrating an axial cross-section view of the sample injection guide embodiment of FIG. 9 taken on the line 12-12 in FIG. 10.

FIG. 13 is a simplified perspective diagram illustrating components of one embodiment of a sample analysis system incorporating a direct sample injection system.

FIG. 14 is a simplified perspective diagram illustrating components of one embodiment of a direct sample injection system.

FIG. 15 is a simplified perspective diagram illustrating additional components of the direct sample injection system of FIG. 14.

FIG. 16 is a simplified flow diagram illustrating the general operation of one embodiment of a method of performing an analysis.

FIG. 17 is a flow chart of steps in multiplexed multitarget screening method, using multiple mutated receptor populations as an example.

FIG. 18 illustrates the Ca²⁺ mobilization response in 20 populations of U937 cells monitored by 3-laser flow cytometry (FCM). Ca²⁺ mobilization was monitored with Indo-1 indicator dye after stimulation of the cells with ATP. Twenty (20) discrete populations were labelled using direct staining with DiO, DiI, DiA, DiD, DiR, FM 1-43, and FM 4-64 fluorochromes. Labelled cells were pooled and internal Ca²⁺ (Ca²⁺ᵢ) levels were analyzed by FCM prior to stimulation with ATP to measure the basal Ca²⁺ᵢ levels, and after stimulation with ATP to measure activation of Ca²⁺ responses (Ca²⁺ᵢ levels) in each population.

FIG. 19 illustrates simultaneous resolution of eight (8) HEP293 cell populations by single cell flow cytometry (FCM), using indirect staining with biotinylated DHPE (N-(biotinoyl)-1,2-dihexadecanoyl-sn-glycero-3-phosphoethanolamine) as an “anchor molecule.” Eight different groups of HEK293 cells were first stained with biotinylated DHPE, then with streptavidin- or avidin-conjugated forms of Alexa 488, Alexa 500, Alexa 514, phycoerythrin, Alexa 594, Alexa 647, Alexa 660 and Alexa 750. The cells were also loaded with the Ca²⁺ᵢ indicator dye Indo-1. Cells were pooled and analyzed by FCM, where cells were exposed to a 488 nm laser and a 647 nm laser to excite the fluorochromes, and to a UV laser source to excite the Indo-1 indicator dye.

FIG. 20 illustrates resolution of ten populations of HEK 293 cells using differential staining with two fluorochromes, a single anchor molecule and a single laser. HEK 293 cells were split into 10 populations, labelled with Biotin-X DHPE as an “anchor molecule”, and then labelled with streptavidin-conjugated Alexa 700 and Streptavidin-conjugated Alexa 635 in the following ratios (SA-Alexa 700:SA-Alexz 635) to achieve differential staining of the populations with the two fluorochromes: Population #1, 0:10; Population #2, 1:9; Population #3, 3:7; Population #4, 1:1; Population #5, 7.5:2.5; Population #6, 9:1; Population #7, 25:1; Population #8, 75:1; Population #9, 10:0; Population #10, unstained (labelled only with Biotin-X DHPE).

FIG. 21 shows the distribution of Ca²⁺ ₁ levels in HEK293 cells transfected with the melanocortin 4 receptor (MC4R), as measured by flow cytometry, at rest (first 20 seconds) and after addition (following the gap) of 1 uM NDP-αMSH, a ligand for the MC4R. The plot shows the results of continuously sampling the population over three minutes; each dot represents a cell, while the solid line represents the average of the sampled population at each bin in the x-axis (time) as calculated from the single cell data.

FIG. 22 shows a response plot of two buffer control samples and an experimental sample containing Indo-1-loaded 5HT2A receptor-bearing cells stimulated with 1 uM serotonin (5HT), using autosampling flow cytometry and single cell analysis to measure Ca²⁺ᵢ levels. Left panel: distribution of Ca²⁺ᵢ levels measured by intensity ratio, and determination of threshold level as shown by arrow. Center panel: flow cytometry results showing the Ca²⁺ᵢ levels (y-axis) of cells from two control populations and one agonist (5HT)-treated population; mean Ca²⁺ᵢ level is shown by the arrow, and two standard deviations above the mean Ca²⁺ᵢ level are also shown. Right panel: bar chart of results from analzying data from plot shown in center panel, showing the percent of cells having Ca²⁺ᵢ at least 2 standard deviations above the mean Ca²⁺ᵢ level in each of the untreated control populations (B1 and B2) had the agonist-treated population (C1).

FIG. 23 shows a comparison of the signal to noise ratio (y-axis) using the mean intensity method and the “all or none” method of measuring the response of 5HT2A receptor-bearing cells to serotonin (5HT), for each concentration of 5HT over a range of 5HT concentrations α-axis).

FIG. 24 shows measurements of apoptosis and necrosis (DAPI fluorescence, upper panel) and proliferative activity (CFDA SE fluorescence, lower panel) in four cell lines: CCRF-CEM (black), RAMOS (dark grey shading), THP-1 (light grey shading), Jurkat cells (white, no shading), in response to exposure to test compounds and controls. Positive and negative controls are shown in the 10 clusters at the right side of the figure, at A3, A6 and A9.

FIG. 25 shows the percentage of CD14+ cells (medium shading), CD4+ cells (lightest shading), and CD14−/CD4− double negative cells (darkest shading) responding to various test compounds and controls. Results for negative controls (buffer controls) are shown at A3 and A4. Results for the ionomycin (Ca²⁺ᵢ ionophore) positive controls shown at B15 and B16. Results using serotonin as a ligand control (positive control) are shown at B3 and B5. Results using test compounds (test ligands) are shown at D9, D10, I7, I8, J9, J10, N7, N8.

### DETAILED DESCRIPTION OF THE INVENTION

The present disclosure provides systems for multiplexed multitarget screening of cell populations having one or more wild type or mutated ligand targets and measuring cell responses to ligands using high throughput screening techniques, including flow cytometry (FCM). The present disclosure provides methods, compositions, apparatus, and analytical techniques for multiplexed multitarget screening. Briefly, the method includes the steps of: 1) developing cell populations to be screened; 2) color-coding cell populations by staining at least one of the cell populations with at least one fluorochrome to yield a distinct excitation/emission optical signature for each cell population; 3) loading cells with one or more indicator dyes to monitor cellular responses; 4) combining stained cell populations into a single mixed cell suspension; 5) analyzing the mixed cell suspension by using light sources to excite each stained cell in the mixed cell suspension and collecting fluorescence emissions from each excited color-coded cell; 6) resolving the individual cell populations present in the mixed cell suspension, based on the distinct optical signature corresponding to each cell population; and 7) deconvoluting data to extract meaningful information about each cell population. The invention includes compositions, apparatus, and analytical techniques suitable for carrying out the method provided herein.

The present disclosure provides systems that are useful for measuring the interaction between a ligand target and a ligand. In particular, the present disclosure provides systems that are useful for measuring the interaction between a ligand target and a ligand. “Ligand target” or “target of interest” is intended to mean any ligand-binding protein including, but not limited to, receptors, ligand-gated ion channels, ligand-binding proteins having a single transmembrane domain, ligand-binding proteins that translocate across a cell membrane after binding, ligand-binding enzymes, ligand-binding regulators of gene expression, ligand-binding structural proteins, or any other protein wherein ligand binding triggers a cellular response. Generally, ligand targets or targets of interest are receptors, including but not limited to receptors involved in signal transduction or signal reporting, in particular, G-protein-coupled receptors (GPCRs). Ligand targets to be screened may be wild type or mutated targets, e.g., wild type receptors or mutated variant receptors. In accordance with various aspects of the invention, these targets may be endogenously expressed, up-regulated endogenous receptor, or receptors expressed as a result of the introduction of exogenous sequences, e.g., receptors expressed by means of regulatable or non-regulatable transfection and expression.

“Ligand” is used to mean any compound being tested in the system disclosed herein, where it is understood that the term generally refers to any ligand that may bind the ligand targets being screened. The term “ligand” as used herein, is intended to be interchangeable with the term “test compound.” Ligands may be natural ligands, modified ligands, mutated ligands, synthetic compounds that are not naturally occurring, synthesized versions of naturally occurring ligands, or any test compound(s) suitable for use as a test compound in the screening system provided herein. Also included in the definition of “ligand” are biological molecules such as peptides, proteins including antibodies, RNA molecules such as antisense RNAs and silencing RNAs, DNA, synthetic nucleotides such as PNA and LNA, aptamers, oligonucleotides, and oligonucleosides. It is further understood that the term “ligand” may be used to refer to a compound that interacts with a ligand and/or a ligand target, or with a ligand-ligand target complex, in such a way as to modulate the biological response to the interaction of the ligand and ligand target.

“Control” or “control compound” is used to mean a treatment used to provide baseline values for cellular reponses. These baseline values are used for comparison purposes in evaluating cellular responses in response to the interaction of a ligand target and ligand. A “control” or “control compound” can be a negative control, most commonly a “buffer control” in which cells containing indicator dye are treated only with the buffer in which the ligand is normally suspended. It is understood that a buffer control may include, in addition to buffering agents, other compounds that are normally found in the solution of ligand being evaluated in a particular embodiment including, but not limited to, solvents, salts, preservatives, antibiotics, or stabilizers. It further understood that the terms “control” or “control compound” encompass embodiments in which cells are not treated with any exogenous compound, e.g., to provide a baseline level of cellular response to compare with both the level of cellular response to the ligand and the level of cellular response to the “buffer control.” A “control” or “control compound” can be a positive control, most commonly a treatment that is known to trigger the cellular response in the cell populations being screened. A positive control can be known ligand of the ligand target on the cell population being screened. A positive control can be a treatment that elicits the cellular response without the involvement of the ligand target, e.g., a calcium ionophore can be used as a positive control in an embodiment that measures Ca²⁺ mobilization as the cellular response. The present disclosure provides non-limiting examples of positive and negative controls for multiplexed multitarget screening. It is understood that one of skill in the art can select one or more control compounds that are appropriate for a particular embodiment.

Cellular responses triggered by the interaction between a ligand target and a ligand include, but are not limited to, changes in ligand target structure or function, ion fluxes across cell membranes, in particular ion fluxes that alter intracellular Ca²⁺ (Ca²⁺ᵢ) levels or cause a change in membrane potential, changes in cell size or shape, cell proliferation, cell differentiation, modulation of the activity of enzymes, alteration in genetic expression, and cell death. Methods for measuring cellular responses are provided that are suitable for use in the screening systems of the present invention.

It is understood that the choice of terms such as “ligand target” and “ligand” are not intended to limit the scope of ligand-binding proteins or ligands that are suitable for screening using the systems provided herein. It is further understood that the choice of the term “cellular response” is not intended to limit the scope of biological responses that are suitable for screening using the systems provided herein.

“Flow cytometer” and “flow cytometry” as used herein, refers to well-known methods and tools described in numerous US patents and scientific references, inter alia, U.S. Pat. No. 3,826,364; U.S. Pat. No. 3,826,412; U.S. Pat. No. 4,600,302; U.S. Pat. No. 4,660,971; U.S. Pat. No. 4,661,913, U.S. Pat. No. 4,988,619; U.S. Pat. No. 5,092,184; U.S. Pat. No. 5,994,089; U.S. Pat. No. 5,968,738; U.S. Pat. No. 6,014,904; U.S. Pat. No. 6,248,590; U.S. Pat. No. 6,256,096; U.S. Pat. No. 6,664,110; U.S. Pat. No. 6,680,367; Haynes, “Principles of Flow Cytometry” (1988) Cytometry Supplement 3:7-18; Ormerod (ed.), Flow Cytometry: A Practical Approach, Oxford Univ. Press (1997); Jaroszeski et al. (eds.), Flow Cytometry Protocols, Methods in Molecular Biology No. 91, Humana Press (1997); and Shapiro, Practical Flow Cytometry, 3rd Ed., Wiley-Liss (1995). The term “FACS” as used herein, refers both to a fluorescence-activated cell sorter, an instrument based on flow cytometry that can select one cell from thousands of other cells, and to the method of fluorescence-activated cell sorting. All remaining terms have their usual meaning in the flow cytometric arts, as set forth, inter alia, in Haynes, “Principles of Flow Cytometry” (1988) Cytometry Supplement 3:7-18; Ormerod (ed.), Flow Cytometry: A Practical Approach, Oxford Univ. Press (1997); Jaroszeski et al. (eds.), Flow Cytometry Protocols, Methods in Molecular Biology No. 91, Humana Press (1997); and Shapiro, Practical Flow Cytometry, 3rd Ed., Wiley-Liss (1995).

“Multiplexed” as used herein refers to simultaneous or near-simultaneous measurement of a plurality of signals. Multiplexed assays as provided herein permit the determination of a plurality of variables in a complex cell sample, e.g., signals can be measured in mixtures containing multiple cell populations bearing multiple ligand targets, and each cell population can be distinguished from the other cell populations in the mixture due to color-coding that yields a distinct optimal signature for each population (see below). Likewise, a plurality of cellular responses can be measured in a complex cell sample, e.g. measurements of ligand binding to ligand targets can be measured simultaneously with changes in Ca²⁺ᵢ levels and membrane potential, due to distinct color-coding of the means for measuring different cellular responses. Multiplexing in the present screening system is further supported by the multiparametric capacity of flow cytometry systems, which allows cells to be identified and distinguished (and, optionally, to be sorted and recovered) on the basis of a plurality of physical, chemical, and biological characteristics.

The multiplexed multitarget screening system provided herein can be considered a “high throughput” screening system, as it allows rapid screening of hundreds or thousands of samples (cell populations, ligand targets, ligands). The screening system provided herein can also be considered a “high content” screening system, as it allows detailed information about the kinetics, specificity, and other temporal-spatial dynamics of the ligand target-ligand interactions being measured. In accordance with one aspect, the information gained from each cell used in the screening system provided herein has such high information content that each cell can be considered a single “experiment” that can easily be repeated using other cells.

It is understood that the present disclosure uses the term “steps” merely to facilitate understanding of various aspects of the invention, and the term is not intended to limit the invention to practicing these so-called “steps” as discrete activities or in a fixed order. Likewise, the presentation of discrete elements of the invention in a certain order in the claims is not intended to limit the invention to practicing these elements as discrete activities or in a fixed order. As described below, the “steps” of the invention can be combined in various ways, and in various orders, without departing form the scope of the present invention.

It is understood that the use of the term “a” or “an” in the specification and claims encompasses both the singular and the plural. Thus, “a” or “an” is intended to have a scope similar to “at least one.”

1. Developing Cell Populations to be Screened.

In accordance with one aspect of the present invention, a first step involves developing one or more cell populations bearing ligand targets, where the cell populations bearing ligand targets are to be screened for their interactions with ligands. In accordance with one aspect, cellular responses of cell populations bearing ligand targets are measured, in order to measure interactions between ligand targets and ligands. Measuring cellular responses triggered by interactions between ligands and ligand targets provides a multiplexed screening method that allows identification of those ligands and ligand targets that interact, and further allows measurements of the affinity and efficacy (amount of cellular response) of the interaction. In certain embodiments, cells bearing wild type ligand targets are screened for their interactions with ligands. In certain embodiments, cells bearing variant or mutant ligand targets are screened for their interactions with ligands.

“Wild type” refers to an existing version of the ligand target. “Variant” is used to refer to a ligand target that differs from the ligand target that has been identified as the wild type for that ligand target. As used herein, “variant” can refer to a naturally occurring variant, e.g., an allele, a single nucleotide polymorphism (SNP), or a splice variant of the gene encoding the wild type ligand target, or to a variant created by deliberate modification or mutation of the ligand target, typically by mutating the DNA encoding the target, but also by chemical modifications of the protein or other alternative methods to modify the final chemical nature of the target. “Mutant” refers to a ligand target encoded by DNA that has been deliberately modified or mutated to encode a mutant ligand target that differs from the wild type ligand target. The terms “variant” and “mutant” are often used in combination in the present disclosure, to encompass all ligand targets that differ from the wild type ligand target. The term “DNA encoding a ligand target” is intended to encompass a scope similar to “ligand target gene” referring both to DNA sequence that is transcribed and translated into the ligand target protein (the coding region) and to associated regulatory regions that may or may not be transcribed or translated.

Variant or mutant ligand targets are generated using standard molecular biology techniques to mutate or otherwise modify DNA encoding the ligand target, where the DNA is modified at one or more residues. Mutations or modifications of DNA encoding the ligand target include, without limitation, additions, deletions, substitutions, duplications, and rearrangements, further including engineered splice variants. DNA encoding a ligand target can be complementary DNA (cDNA) reverse-transcribed from messenger RNA (mRNA), or synthesized DNA. In accordance with the present invention, both coding and non-coding (regulatory) regions of DNA can be modified or mutated to produce a mutant ligand target. Standard techniques for modification or mutation of DNA to generate mutated ligand targets include “shotgun” mutagenesis, cassette mutagenesis, chemical mutagenesis, site-directed mutagenesis, in situ mutagenesis, mutator strain induced mutagenesis, RNA-DNA chimeroplasty for targeted mutagenesis, DNA shuffling, error-prone PCR, other combinatorial techniques, or other standard techniques as found, for example, in Sambrook et al., Molecular Cloning, A Laboratory Manual, (3^{rd} Ed. (2000), 2^{nd} Ed. (1989), Cold Spring Harbor Laboratory Press, N.Y., or Ausubel et al., Eds. Current Protocols in Molecular Biology, (1991 and updates) Wiley Interscience, N.Y.

Ligand targets may be expressed from DNA already present in a cell, or may be expressed from DNA that has been introduced into a cell. Wild type, variant, and mutant ligand targets may be expressed from DNA already present in a cell. In one non-limiting example, a mutant ligand target is expressed from mutated DNA already present in a cell, where the cell has been subjected to chemical mutagenesis. In accordance with this aspect, the expression of ligand targets is regulated by endogenous regulatory elements such as promoters, enhancers, activators, or repressors. In one embodiment, ligand targets expressed from DNA already present in a cell are constitutively expressed under the control of constitutive promoters or enhancers. In another embodiment, expression of ligand targets expressed from DNA already present in a cell is under the control of inducible regulatory elements, e.g., inducible promoters or repressors, and expression depends on manipulation of these regulatory elements.

Wild type, variant, and mutant ligand targets may be expressed from DNA that has been introduced into a host cell. The term “transfection” is intended to include any means by which a nucleic acid molecule can be introduced into eukaryotic or prokaryotic cells. The introduced nucleic acid molecule can be DNA or RNA, and may be either single or double-stranded; in the present disclosure, the introduced nucleic acid molecule is referred to as DNA. As used herein, “transfection” encompasses both transient cell transfection, wherein the DNA encoding a ligand target is transiently expressed, and stable transformation of cells, wherein the DNA encoding a ligand target is maintained by integration into chromosomal DNA or persistence in a stable extrachromosomal element. DNA used in transfection of host cells can be circularized, e.g., in a vector (plasmid) or may be linear, depending on the transfection and expression method selected for a particular embodiment. “Recombinant” expression of ligand targets refers to transfection of host cells and expression of the introduced DNA encoding ligand targets.

In accordance with one aspect, a host cell is transfected with DNA encoding a wild type ligand target, producing a cell bearing a recombinantly expressed wild type ligand target. In accordance with another aspect, a host cell is transfected with DNA encoding a variant or mutant ligand target, producing a cell bearing a recombinantly expressed variant or mutant ligand target.

Suitable transfection methods include, but are not limited to, a variety of techniques useful for introduction of nucleic acids into mammalian cells including electroporation, calcium phosphate precipitation, DEAE-dextran treatment, lipofection, microinjection, and viral infection. Suitable methods for transfecting mammalian cells can be found in Sambrook et al., Molecular Cloning, A Laboratory Manual, (3^{rd} Ed. (2000), 2^{nd} Ed. (1989), Cold Spring Harbor Laboratory Press, N.Y., or Ausubel et al., Eds. Current Protocols in Molecular Biology, (1991 and updates) Wiley Interscience, N.Y. and other laboratory textbooks. In accordance with one aspect, non-viral-mediated methods for introducing DNA into a host cell include use of a cell-delivery vehicle such as cationic liposomes or derivatized (e.g., antibody conjugated) polylysine conjugates, gramicidin S, or artificial viral envelopes, e.g. as described in Philip et al, (1994) Mol Cell Biol 14:2411. In another embodiment, DNA encoding a ligand target is delivered into a host cell in the form of a soluble molecular complex including a nucleic acid binding agent and a cell-specific binding agent, such that the complex binds to the host cell surface and is subsequently internalized by the cell, e.g., as described in U.S. Pat. No. 5,166,320. In another embodiment, DNA encoding a ligand target is introduced into a host cell by particle bombardment, as described in Yang and Sun (1995) Nature Medicine 1:481. In accordance with another aspect, DNA encoding ligand targets can be introduced using vectors, e.g., viral vectors including but not limited to recombinant retroviruses, adenovirus, adeno-associated virus, and herpes simplex virus-1.

One suitable transfection method is known as Single Target Integration Site (STIS), wherein only one DNA sequence is successfully transfected into, and expressed by, each host cell, and each DNA is integrated into the identical genomic locus in each cell using homologous recombination technology. This technique ensures that the expression of each integrated DNA is consistent from cell to cell within the population of cells. In one embodiment, STIS is selected to ensure homogeneity of assay results.

Another suitable transfection method is Random Target-Integration Site (RTIS). A standard transfection technique such as lipofectamine or electroporation, is used to transfect a cell population. It should be noted that, since there is no assurance that each cell integrates the transfected DNA at the same location in the genome, RTIS can lead to different expression levels between cells within the population.

It is understood that expression of ligand targets may be affected by designing the DNA molecule to be introduced into host cell, to include various sequences that can regulate expression of DNA encoding a ligand target. Such a molecule typically contains regulatory elements to which the DNA encoding a ligand target is operably linked in a manner which may influence transcriptional, translational, or post-translational events related to expression of the ligand target in host cells. Regulatory elements are selected to direct expression of the ligand target in a suitable host cell and include, but are not limited to, promoters, enhancers, polyadenylation signals, and sequences necessary for transport of the ligand target to the appropriate cellular compartment (usually, insertion into the cell membrane). When the introduced DNA is a cDNA in a recombinant expression vector, the regulatory element controlling transcription and/or translation of the cDNA are often derived from viral sequences. Regulatory elements are known in the art and are described in, e.g., Goeddel, Gene Expression Technology: Methods in Enzymology 185, Academic Press, San Diego, Calif. (1990).

Regulatory sequences linked to the DNA encoding the ligand target include promoters that can be selected to provide constitutive or inducible transcription. Suitable promoters for use in various systems are known in the art. For example, suitable promoters for use in murine cells include RSV LTR, MPSV LTR, SV40 IEP, and metallothionein promoter, and CMV IEP is a suitable promoter for use in human cells. Examples of commonly used viral promoters include those derived from polyoma, Adenovirus 2, cytomegalovirus and Simian Virus 40, and retroviral LTRs.

In a specific embodiment, DNA encoding the ligand target is under the control of an inducible control element such as an “inducible promoter” or enhancer, such that expression can be regulated by contacting (or not contacting) the host cell with an agent which affects the inducible control element. Inducible regulatory systems for use in mammalian cells are known in the art, for example systems in which gene expression is regulated by heavy metal ions (Mayo et al., (1982) Cell 29:99-108; Brinster et al., (1982) Nature 296:39-42; Searle et al., (1985) Mol Cell Biol 5:1480-1489), heat shock (Nouer et al. (1991) in Heat Shock Response, Nouer, ed., CRC, Boca Raton, Fla., pp167-220), hormones (Lee et al. (1981) Nature 294:228-232; Hynes et al. (1981) Proc. Natl. Acad. Sci. USA 78:2038-2042; Klock et al. (1987) Nature 329:734-736; Israel & Kaufman (1989) Nuc. Acids Res. 11:2589-2604 and PCT Publication No. WO 93/23431), tetracycline (Gossen, M. and Bujard, H. (1992) Proc. Natl. Acad. Sci. USA 89:5547-5551 and PCT Publication No. WO 94/29442) or FK506 related molecules (PCT Publication No. WO94/18317).

In another embodiment of the invention, DNA encoding a ligand target is under the control of regulatory sequences such as “constitutive promoters” or constitutive enhancers which constitutively drive the expression of the DNA encoding a ligand target Exemplary constitutive promoters include, but are not limited to, the promoters for the following genes: hypoxanthine phosphoribosyl transferase (HPRT), dihydrofolate reductase (DHFR) (Scharfmann et al., Proc. Natl. Acad. Sci. USA 88: 4626-4630 (1991)), adenosine deaminase, phosphoglycerol kinase (PGK), pyruvate kinase, phosphoglycerol mutase, the β-actin promoter (Lai et al., Proc. Natl. Acad. Sci. USA 86: 10006-10010 (1989)), and other constitutive promoters known to those of skill in the art. In addition, many viral promoters function constitutively in eucaryotic cells including, but not limited to, early and late promoters of SV40 (See Bernoist and Chambon, Nature, 290:304 (1981)); long terminal repeats (LTRs) of Moloney Leukemia Virus and other retroviruses (See Weiss et al., RNA Tumor Viruses, Cold Spring Harbor Laboratory, Cold Spring Harbor, N.Y. (1985)); thymidine kinase (TK) promoter of Herpes Simplex Virus (HSV) (See Wagner et al., Proc. Nat. Acad. Sci. USA, 78: 1441(1981)); cytomegalovirus immediate-early (IE1) promoter (See Karasuyama et al., J. Exp. Med., 169: 13 (1989); Rous sarcoma virus (RSV) promoter (Yamamoto et al., Cell, 22:787 (1980)); adenovirus major late promoter (Yamada et al., Proc. Nat. Acad. Sci. USA, 82: 3567 (1985)), and other viral-derived constitutive promoters known to those of skill in the art

Host Cells

It is understood that suitable host cells may be chosen according to the characteristics of each embodiment. In accordance with one aspect, suitable host cells for transfection with DNA encoding a ligand target are cells that do not normally express the ligand target. In these cells, the cellular responses that are measured are considered to reflect the interaction of the ligand target with the ligand.

In accordance with another aspect, host cells that normally express the wild type ligand target are transfected with DNA encoding a variant or mutant ligand target. In host cell that normally express the ligand target, the baseline cellular responses characteristic of the normal cell expressing a wild-type ligand target are known, such that differences from that baseline may be ascribed to the effect of also expressing a variant or mutant ligand target. It is understood that choice of host cell may be determined by choice of expression vector. Cultured cells suitable for use as host cells may be tranformed (transfected) and/or immortalized. Examples of suitable host cells include, but are not limited to, HEK293, U937, COS-7, NIH/3T3, HeLa, CHO, CCRF-CEM, Jurkat, RAMOS, and THP-1 cell lines.

In certain embodiments, non-mammalian host cells may be suitable, including but not limited to, Drosophila melanogaster S2 cells, Spodoptera frugiperda Sf9 cells, High-5 cells, yeast cells including Saccharomyces species or Pichia species, and bacterial cells e.g., E. coli.

The suitability of a particular cell for use as a host cell in accordance with the invention will depend on the ability to introduce a DNA encoding a ligand target into the cell, and express the ligand target. Cells may be adherent or non-adherent. Host cells may be chosen or developed on the basis of certain desirable properties of the precursor cells of the host cells. It is understood that one of skill in the art will select a suitable host cell according to goals, characteristics, conditions, and/or constraints of a particular embodiment.

In accordance with another aspect, host cells that already express a ligand target may be treated to induce expression of a variant or mutant ligand target without transfection with DNA encoding a ligand target. One suitable method involves in situ mutagenesis of a host cell expressing a ligand target, e.g., by chemical or radiation mutagenesis. Another suitable method involves insertional mutagenesis, e.g., transposon mutagenesis, of a host cell expressing a ligand target. In one embodiment, the host cell already contains one or more transposons that are activated. In another embodiment, one or more transposons are introduced into the host cell. After mutagenesis, cells are screened to identify those cells expressing variant or mutant ligand targets.

Host cells may also include normal cells such as blood cells or primary tissue isolates. Blood cell types suitable for use as host cells include, but are not limited to, peripheral blood cells, in particular peripheral blood mononuclear cells (HPBMC), basophils (polymorphonuclear basophils, PMBs), eosinophils (polymorphonuclear eosinophils, PMEs), lymphocytes, monocyte, neutrophils (polymorphonuclear neutrophils, PMNs), platelets (thrombocytes), or red blood cells (erythrocytes; where it is understood that erythrocytes expressing ligand targets are expressing endogenous ligand targets, or that transfection or mutagenesis to induce expression of variant ligand targets was carried out using erythrocyte precursors prior to enucleation). Such cells may be freshly isolated and immediately prepared for multiplexed analysis, or isolated, placed in tissue culture for a period of time and then prepared for analysis. In one embodiment,

Cell Populations

The present invention provides drug-target-expressing cell populations for use in the multiplexed multitarget screening method disclosed herein. Cells expressing ligand targets of interest are produced and identified, e.g., by any of the methods described above, and cell populations are developed from these cells. For each embodiment, cell populations are develped using methods suitable for the host cell(s) used in the embodiment. Briefly, cell populations are developed by incubating cells with one or more agents sufficient to stimulate cell division and proliferation in culture, e.g., by adding cytokines, growth factors, and nutrients, under conditions which also facilitate expression of the ligand target(s) by the cells. The term “cell expansion” is often used in the art to describe the process of stimulating cell proliferation to generate a suitable cell population as described, e.g., in Mather and Barnes, Animal Cell Culture Methods, (1998), Academic Press; Harrison et al., General Techniques of Cell Culture (1997) Cambridge University Press; and Doyle et al., Cell and Tissue Culture: Laboratory Procedures (1998) John Wiley and Sons. For cells that express a ligand target under control of an inducible promoter, cell populations are developed in the presence of the inducing stimulus or stimuli. In certain embodiments, stimuli that induce expression of ligand targets are added during the cell expansion process. In other embodiments, stimuli that induce expression of ligand targets are added after an expanded cell population has been obtained.

In accordance with one aspect, cell populations are developed wherein the cells express one wild-type version of one ligand target per cell. Cell populations expressing one wild-type version of one ligand target per cell can be developed by any suitable method including any of the methods described above. In one embodiment, a cell population is developed from a cell that normally expresses a wild type ligand target. In another embodiment, a cell population is developed from a cell that does not normally express a wild type ligand target, and has been transfected with DNA encoding a wild type target. Multiple cell populations can be developed wherein each cell population expresses one wild-type version of one ligand target per cell.

In accordance with another aspect, cell populations are developed wherein the cells express one variant of one ligand target per cell. In accordance with one aspect, the variant is a naturally-occurring variant of the wild type ligand target. In accordance with another aspect, the variant is a mutant variant of the ligand target. Cell populations expressing one variant of one ligand target per cell can be developed by any suitable method including any of the methods described above. Multiple cell populations can be developed wherein each cell population expresses one variant of one ligand target per cell.

In accordance with one aspect, cell populations are developed wherein the cells express more than one ligand target per cell. Cell populations expressing more than one ligand target per cell can be developed using any suitable method including any of the methods described above. A cell population may be transfected with more than one DNA sequence encoding more than one ligand target. In certain embodiments, a cell population contains cells that express more than one wild-type ligand target per cell. In other embodiments, cell populations contain cell that express more than one variant ligand target per cell. In one embodiment, cell populations contain cell that express more than one naturally occurring variant ligand target per cell. In one embodiment, cell populations contain cell that express more than one mutant variant ligand target per cell. In one embodiment, cell populations contain cell that express one or more naturally occurring variant ligand targets and one or more mutant variant ligand targets per cell. In one embodiment, cell populations contain cells that express both wild-type and variant ligand targets per cell. In certain embodiments, cell populations contain cells that express both wild-type and variant ligand targets per cell. In one embodiment, cell populations contain cells that express both wild-type and naturally occurring variant ligand targets per cell. In one embodiment, cell populations contain cells that express both wild-type and mutant variant ligand targets per cell. In one embodiment, cell populations contain cells that express wild-type ligand targets, naturally occurring variant ligand targets, and mutant variant ligand targets in the same cell.

In accordance with another aspect of the present invention, an expressed ligand target includes an optional expression tag or epitope tag recognized by antibodies or other binding materials. It is understood that suitable expression tags (epitope tags) will not interfere with the ligand-binding properties or other functional activities of the ligand target. Expression tags can be used to isolate recombinantly expressed proteins from host cells, allowing purification, sequencing, or further study of the isolated proteins. Expression tags can be used to identify cells having specific ligand targets, especially when a cell population contains more than one wild-type or mutated variant ligand target. Using known expression tags allows convenient purification using known methods and materials, thereby eliminating the need to develop antisera or purification strategies that are specific for individual proteins. Suitable expression tags include, but are not limited to, antigen (epitope) tags such as FLAG, c-MYC, HSV, V5, HA, hexahistidine (HIS), or others that can be identified by one of skill in the art.

2. Color-Coding Cell Populations to Yield a Distint Optical Signature for Each Population.

In accordance with another aspect of the invention, a second step involves color-coding cell populations to yield a distinct optical signature for each cell population. Color-coding cell populations as provided herein includes staining cell populations with fluorochromes to develop color-coded populations suitable for single cell analysis, e.g., by single cell flow cytometry. Color-coding cell populations as provided herein further includes not staining a cell population to yield a distinct optical signature for the unstained cell population, as illustrated in Example 6, below.

Individual fluorochromes and/or combinations of fluorochromes are used to stain cell populations to yield a unique “signature” for each population that can be resolved based on excitation source and emission profile. Regardless of the transfection or mutagenesis methodology used to achieve ligand target expression, each cell population expressing one or more ligand targets is grown separately in cell culture and individually color-coded such that any cell of the population can be discretely recognized by its “signature” excitation/emission profile. In one embodiment, each cell population expressing one ligand target is grown separately in cell culture and individually color-coded. In another embodiment, a cell population expressing more than one ligand target is grown in cell culture and color-coded, such that the ‘signature’ of that population is correlated with more than one ligand target. In various embodiment, a color-coded population may have 1, 2, 3, 4, 5, 6, 7, 8, 9, 10, 11, 12, 13, 14, 15, 16, 17, 18, 19, 20, 21, 22, 23, 24, 25, 30, 35, 40, 45, 50 or more ligand targets.

“Color-coding” refers to the process of staining each cell population with individual fluorochromes, or fluorochrome combinations, that have emission spectra that can be distinguished from other fluorochromes or fluorochrome combinations, yielding a distinct “optical signature” for each population based on the excitation source and emission profile of the fluorochrome(s) used. Each fluorochrome has a distinct pair of excitation and emissions spectra, with unique optima and absorption strengths and emission intensities throughout the excitation and emissions spectra. When a specific excitation laser source that emits a unique band of light is used to excite a color-coded cell, the cell emits a particular fluorescent pattern of light that can be detected and deconvoluted by fluorescence emissions acquisition hardware and software. When multiple stained populations are pooled into a single population for the analysis, each population having a distinct optical signature as a result of single or multiple staining, it is possible to recognize the discrete staining pattern (the “optical signature”) of each population by analyzing the pooled population with a single or multi-laser FCM. The diversity of optical signatures for cell populations of the present invention results from utilizing different fluorochromes and combinations of fluorochromes, as well as from utilizing different ratios of fluorochromes in combination, and different concentrations (intensities) of fluorochromes.

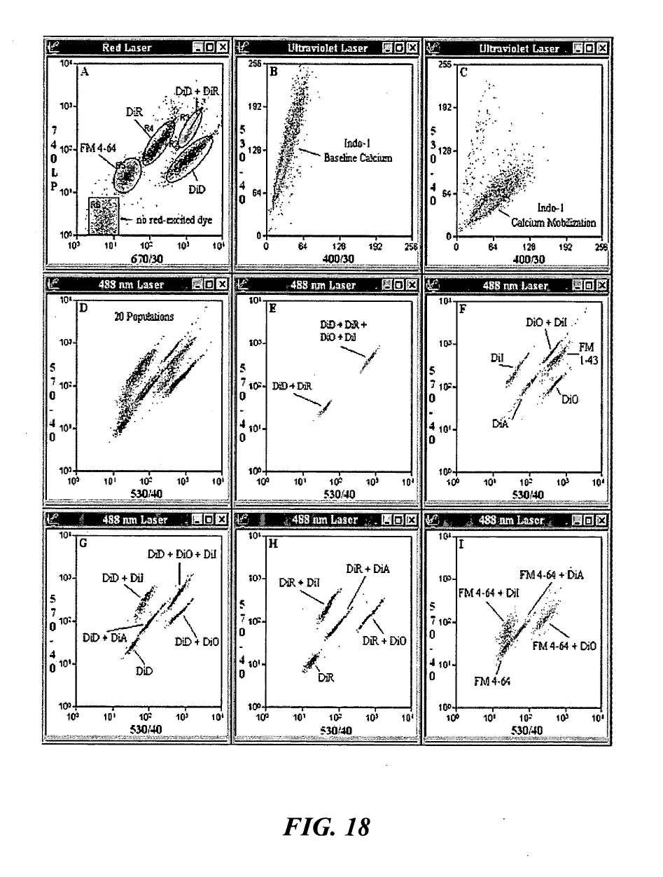

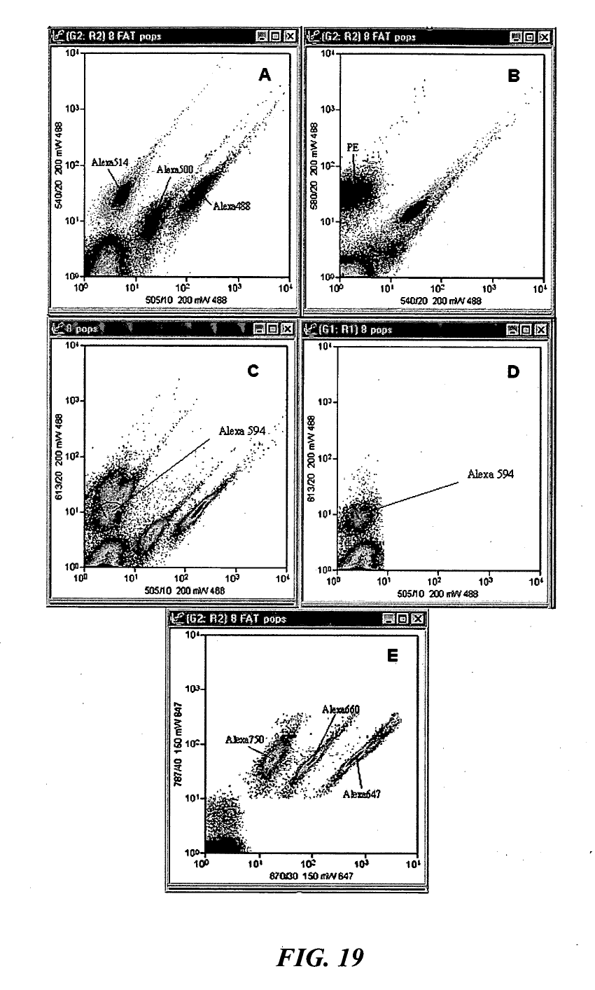

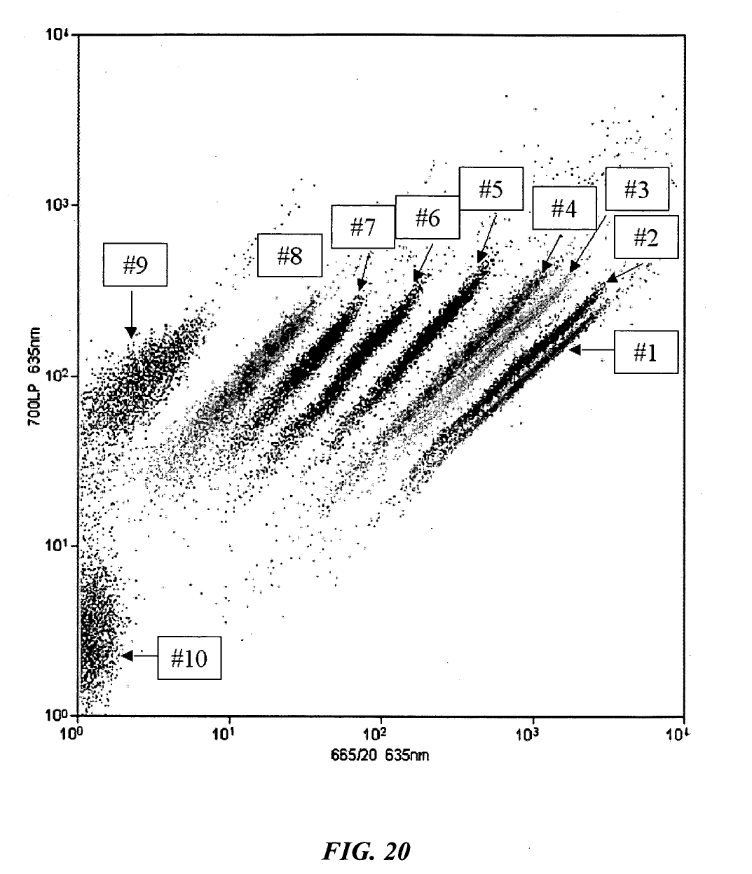

The number of distinct populations that can be resolved as provided herein is increased with additional lasers and emission detection pathways. A single laser system may be suitable for resolving mixtures containing 3-4 cell populations, while a three-laser system may resolve 20 or more cell populations, as illustrated in FIGS. 18 and 19.

Generally, the color-coding of each population is achieved by staining each cell population separately, after which the stained cells from these populations can then be mixed. Color-coded (stained) cell populations can then be mixed and exposed to the same ligand (ligand), and the cellular response of each cell in each population can be measured individually. Cells can be directly stained with fluorochrome labels, or fluorochrome labels can be attached to “anchor” molecules that are attached to cells. Cells can be color-coded using fluorochrome-conjugated antibodies specific for expression tags on the expressed ligand targets. Suitable staining techniques are those wherein: (1) the probes label the cells in each stained population to a homogeneous and uniform state; (2) the probes do not interfere with the biological readout or parameters being investigated in all the populations; and (3) the probes remain in the cells long enough to enable a homogeneous staining pattern. It is understood that methods of staining cells is not limited to the recited methodologies, and that one of skill in the art can adapt other methods to color-code cells for use as provided herein.

Direct Staining with Fluorescent Hydrophobic Labels (Fluorochromes).

In accordance with one aspect of the invention, cells expressing one or more ligand targets can be directly stained using fluorescent hydrophobic molecules (fluorochromes) to directly stain cells. Analysis of a mixture of multiple populations of cells stained using this method is illustrated in FIG. 18.

Suitable fluorochromes include, but are not limited to, fluorescent carbocyanine probes, dialkylcarbocyanine and dialkylaminostyryl probes, or other hydrophobic (lipophilic) fluorochromes such as FM 1-43 (Invitrogen, Carlsbad Calif.; formerly from Molecular Probes, Inc., Eugene, Oreg.) or the PKH-2 or PKH26 probes (Sigma-Aldrich, St. Louis, Mo.) that can label cells in vitro or in vivo for days to weeks. Other lipophilic fluorochromes such as Bodipy 665 can also be used even though these fluorochromes are not specifically manufactured for cell labelling. Other probes that label specific organelles or cellular compartments such as the LysoSensor Blue (Molecular Probes, Inc.) or the DNA stain Hoechst 33342 can also be used as long as they are retained within the cells for hours and do not interfere with the cellular response to be studied in the screen. The present disclosure further provides fluorochromes that label reactive groups on cell surfaces or inside cell interiors, in particular amine-reactive fluorchromes available from Invitrogen (Carlsbad, Calif.) including, but not limited to, succimidyl ester (SE) probes available from Invitrogen (Carlsbad Calif.). Additional suitable fluorochromes for use in the invention include, but are not limited to, fluorescent lanthanide complexes, including those of Europium and Terbium, fluorescein, rhodamine, tetramethylrhodamine, eosin, erythrosin, coumarin, methyl-coumarins, pyrene, Malachite green, stilbene, Lucifer Yellow, Cascade Blue™, Texas Red, and others described by Haugland, The Molecular Probes Handbook of Fluorescent Probes and Research Chemicals 6^{th} Ed. (1996), Molecular Probes Inc., Eugene Oreg. (Also available as The Handbook of Fluorescent Probes and Indicators, 9^{th} Ed., available online as “The Handbook, Web Edition” at http://www.probes.com/handbook).

Additional labels include cyanine fluorochrome complexes as described in U.S. Pat. No. 6,673,943. Briefly, such a complex includes a first, or donor, fluorochrome having first absorption and emission spectra, and a second, or acceptor, fluorochrome having second absorption and emission spectra, wherein at least one of the first or second fluorochromes is a cyanine dye. The wavelength of the emission maximum of the second fluorochrome is longer than the wavelength of the emission maximum of the first fluorochrome, and a portion of the absorption spectrum of the second fluorochrome overlaps a portion of the emission spectrum of the first fluorochrome for transfer of energy absorbed by the first fluorochrome upon excitation with light to the second fluorochrome. The complex also includes a linker for covalently attaching the fluorochromes to permit resonance energy transfer between the first and the second fluorochromes, while separating the fluorochromes by a distance that provides efficient energy transfer. These fluorochrome complexes can include a group capable of attaching to an “anchor molecule” for use in the indirect staining method described below. The labeling complexes of the invention are synthesized preferably by covalently linking cyanine fluorochromes to other cyanine flurorochromes to form energy donor-acceptor complexes. Cyanine fluorochromes are particularly useful for preparation of these complexes because of the wide range of spectral properties and structural variations available. See, for example, Majumdar et al., “Cyamine dye labeling reagents. Sulfoindocyanine succininmidyl ester” Bioconjugate Chemistry, 4:105-111 (1993) and U.S. Pat. No. 5,268,486 to Waggoner et al., the disclosure of which is incorporated herein by reference.

Additional labels include nanocrystals or Q-dots as described in U.S. Pat. No. 6,544,732.

One of skill in the art can select one or more fluorochromes suitable for a particular embodiment using guidance provided, e.g., at Haugland, The Molecular Probes Handbook of Fluorescent Probes and Research Chemicals 6^{th} Ed. (1996) Molecular Probes Inc., Eugene Oreg., and related publications.

In accordance with one aspect, stock solutions of the fluorochromes are prepared using suitable solvents, and are added to cells in a staining solution. In non-limiting exemplary embodiments, the probes are solubilized in dimethyl sulphoxide (DMSO), dimethylformamide (DMF) or ethanol at 1-10 mM to provide stock solutions. In one embodiment, the cells are suspended in a suitable buffer such as Hybridoma Medium (Sigma-Aldrich), and each cell population is stained separately by diluting the cells to a concentration between about 1×10⁵ to 1×10⁶ cells/ml and adding the fluorochrome stock solution(s) to each solution of cells in buffer, to a final concentration of 1-10 uM. Staining solutions may contain, in addition to cells, fluorochrome(s), and buffer, other ingredients that facilitate staining of the cells. It is understood that the concentration and amounts recited herein are non-limiting and are for purposes of guidance only. One of skill in the art can apply the teachings provided herein to determine the optimal concentrations of cells, fluorochrome(s), and other components for any particular embodiment.

Two-Step, or Indirect, Staining Using Anchor Molecule and Fluorochromes.

The present disclosure also provides a two-step, or indirect, staining procedure wherein the cells expressing one or more ligand targets are first labelled by a non-fluorescent “anchor molecule” and fluorochromes are then conjugated to the anchor molecule. In accordance with this aspect, an aliquot of anchor molecule stock solution is added to a cell suspension in a suitable medium, to a final concentration sufficient to label cells. A cell suspension typically has from about 1×10⁶ to 10×10⁶ cells per milliliter (ml). In certain embodiments, in particular for light-sensitive compounds, it is desirable for cells mixed with anchor molecule to be kept in dark conditions, e.g., wrapped in foil. The cells and anchor molecule(s) are mixed, e.g. on a shaker or rotator platform. Cells can be labelled for a suitable time depending on temperature, cell concentration, anchor molecule concentration, and affinity of labelling. In one embodiment, biotinylated phosphoethanolamine (biotinylated DHPE, Invitrogen, Carlsbad Calif.) is used as an anchor molecule. Analysis of a mixture of multiple populations of cells stained using this two-step methodology is illustrated in FIG. 19. In one embodiment, biotinylated DHPE is added to the cell suspension (at about 1×10⁵ to 1×10⁶ cells/ml) in hybridoma medium, from a stock solution of 1-10 mg/ml, to a final concentration of 0.5 to 10 μg/ml. The cells are wrapped in foil and placed on a rotator platform at room temperature for 30-90 minutes, preferably 60 minutes.

In accordance with one aspect of the invention, cells are loaded with one or more indicator dyes to monitor cellular responses to ligands after the step of labelling with the anchor molecule and before the final labelling step. Suitable methods and compositions for loading cells with indicator dye to monitor cellular responses are disclosed below. In certain embodiments, intracellular Ca²⁺ (Ca²⁺) is monitored as an indicator of mobilization of internal Ca²⁺ stores in response to ligand (ligand) binding, e.g., using Indo-1 or Fluo4 dyes (Invitrogen). In one embodiment, cells are centrifuged after the incubation with the anchor molecule to label the cells, and resuspended in a suitable medium, e.g., in hybridoma medium at concentration of from 1-10×10⁶ cells/ml.

After cells have been labelled with an anchor molecule, a second label that will bind to the anchor molecule is added to the cells. The second label includes any fluorescent molecule that binds to the anchor molecule or that is conjugated to a molecule that binds to the anchor molecule, such that the second label binds to the anchor molecule already attached to the cell membrane and thereby color-code the cell. Optionally, the second label is added after the cells have been labelled with an anchor molecule and have been loaded with an indicator dye to monitor cellular responses. Generally, cells are incubated with a second label for about 30-60 minutes. In embodiments using a biotinylated anchor molecule, the second label generally includes a fluorochrome conjugated to avidin or streptavidin, or to another molecule that can recognize biotin such as an anti-biotin antibody or modified avidin or streptavidin. In one embodiment using biotinylated DHPE as the anchor moleculae, FITC-streptavidin was used as the second label and labelled the cell by binding to the biotinylated DHPE already in the cell membrane.

The second label includes a fluorochrome or combination of fluorchromes that has been selected for each cell population to be stained, to achieve “color coding” and a distinct optical signature for each cell population as described above. In certain embodiments using a biotinylated anchor molecule, suitable fluorochromes include streptavidin conjugates of fluorescein, coumarin or rhodamine are used, including streptavidin- or avidin-conjugated forms of Alexa™ fluorochromes, such as Alexa 488, Alexa 500, Alexa 514, phycoerythrin, Alexa 594, Alexa 647, Alexa 660 and Alexa 750 (Invitrogen). FIG. 19 illustrates results of an embodiment using streptavidin-conjugated fluorochromes. Because virtually any fluorochrome that can be conjugated to an anchor-molecule-binding molecule can be utilized, the indirect staining method provides a wide range of choices for developing unique “signature” absorption and emission spectra. The indirect staining method further provides homogeneous staining of cell populations. For example, any fluorochrome that can be conjugated to avidin or streptavidin can be utilized to stain cells that have already been labelled with a biotinylated anchor molecule. As provided herein, the indirect staining technique has the potential to yield a large number of distinctly resolvable cell populations.

Both the direct and indirect staining techniques provided herein result in color-coded cell populations suitable for use in the present invention when: 1) the probes label the cells in each stained cell population to a homogeneous and uniform state; 2) the probes do not interfere with the cellular responses being monitored in the cell populations; 3) the probes do not interfere with the measurement (or, “readout”) of the cellular responses being monitored in the cell populations; and 4) the probes remain in the cells in a homogeneous pattern for a sufficient amount of time to carry out the analysis. In certain embodiment, the probes remain in the cells for at least about two hours.

Addition of Indicator Dyes to Monitor Cellular Responses

In accordance with another aspect of the present invention, cells of the present invention are loaded one or more additional fluorochromes that act as “indicator dyes” to monitor cellular responses to the exposure of a ligand target to a ligand. As provided herein, cellular responses are measured as changes in cellular physiology including, but not limited to, mobilization of internal Ca²⁺ (Ca²⁺ᵢ) stores, changes in membrane potential, changes in cytoplasmic or intraorganellar pH, and changes in intracellular concentrations of various ions other than Ca²⁺.

“Indicator dyes” as provided herein include nucleic acid stains that indicate relevant cellular reponses, e.g., Hoechst 33342 can be used as a viable DNA stain to monitor a cellular response that may include chromatin fragmentation and/or apoptosis. The present disclosure provides methods for measuring cellular responses can be measured in entire cells or in organelles, e.g., measuring ion concentrations in the cytoplasm or in organelles such as mitochondria, nuclei, chloroplasts, endoplastic reticulum (ER), Golgi apparatus, or proteasomes, and measuring membrane potential across the plasma membrane and/or across organellar membranes.

In accordance with one aspect, one or more indicator dyes are added prior to staining (“color-coding”) of a cell population. In one embodiment, a cell population bearing a ligand target is loaded with an indicator and is then color-coded with a distinct optical signature. In another embodiment, a cell population bearing a ligand target is loaded with an indicator and then divided into sub-populations, each of which is then color-coded with a distinct optical signature. In accordance with one aspect, one or more indicator dyes are added during staining (“color-coding”) of a cell population. In one embodiment, the surface stains and indicator dye are added together to cells in a staining solution. In accordance with another aspect, one or more indicator dyes are added after staining of a cell population. In one embodiment, a cell population is rinsed after color-coding, and then exchanged into a fresh solution for indicator dye loading.

In accordance with one aspect, intracellular Ca²⁺ (Ca²⁺ᵢ) is monitored as an indicator of mobilization of internal Ca²⁺ stores in response to the interaction of the ligand target with the ligand (ligand). In certain embodiments, Ca²⁺ in cells and organelles (e.g., mitochondria) is measured using fluorescent Ca²⁺ indicators as described in The Handbook of Fluorescent Probes and Indicators, 9^{th} Ed., Chapter 20, (Molecular Probes, Invitrogen; available online as “The Handbook, Web Edition” at http://www.iprobes.com/handbook) Suitable Ca²⁺ᵢ indicator dyes include, but are not limited to, Indo, Fluo, BAPTA indicators available from Invitrogen (Carlsbad Calif.), in particular Indo-1, Fluo-3, Fluo-4, Oregon Green 488 BAPTA, Calcium Green, X-rhod-1 and Fura Red indicators and their variants, which allow Ca²⁺ᵢ detection over a wide concentration range. In certain embodiments, fluorescent indicators are conjugated to high- or low-molecular weight dextrans for improved cellular retention and less compartmentalization. In certain embodiments, fluroescent indicators can be conjugated to lipophilic Ca²⁺ indicators for measuring near-membrane Ca²⁺. Suitable methods for loading cells with Ca²⁺ᵢ indicators include using the acetoxy methyl ester (AM) form of the dye at a concentration of from 0.5 to 10 μM, from a stock solution in DMSO, to the cell suspension while the cells are being stained with color-coding dyes. Generally, the total DMSO is kept at about 1% or less by volume in the cell suspension. Generally, cells are wrapped in foil to prevent photobleaching of any of the probes by ambient light. Cells are generally placed on a rotating or rocking platform to keep the cells in suspension. The cells are incubated in suspension at a suitable temperature for a suitable amount of time, often between about 30 to about 90 minutes, typically about 60 minutes. Depending on the particular embodiment, cells may be incubated at room temperature, e.g., 25° C., or at lower or higher temperatures, e.g., 37° C. One of skill in the art can determine suitable labelling and incubation conditions for each particular embodiment.

In accordance with another aspect, the concentrations of other divalent cations are monitored in response to the interaction of a ligand target with the ligand (ligand). In certain embodiments, divalent cations including, but not limited to, Mg²⁺, Zn²⁺, Ba²⁺, Cd²⁺, and Sr²⁺, in cells and organelles (e.g., mitochondria) is measured using fluorescent indicators as described in The Handbook of Fluorescent Probes and Indicators, 9^{th} Ed., Chapter 20, (Molecular Probes, Invitrogen; available online as “The Handbook, Web Edition” at http://www.probes.com/handbook). In accordance with one aspect, zinc concentrations can be measured using fluorescent indicators nominally designed for Ca²⁺ detection such as fura-2, or using fluroescent indicators with greater Zn2+ selectivity, such as FuraZin-1, IndoZin-1, FluoZin-1, FluoZin-2, and RhodZin-1 (all available from Invitrogen), which detect Zn²⁺ in the 0.1-100 μM range with minimal interfering Ca²⁺ sensitivity, or using Zn²⁺ indicators that have essentially no sensitivity to Ca²⁺, e.g., Newport Green DCF and Newport Green PDX (available from Invitrogen). The spectral responses of these indicators closely mimic those of the similarly named Ca²⁺ indicators, e.g., FuraZin-1 and IndoZin-1 exhibit Zn²⁺-dependent excitation and emission spectral shifts, respectively, and FluoZin-2 and RhodZin-1 show Zn²⁺-dependent fluorescence without accompanying spectral shifts (see, Handbook of Molecular Probes, supra, Chapter 20). It is understood that the “color-coding” aspect of the present invention allows cells containing the above-mentioned zinc indicators to be analyzed in a mixed cell suspension containing, e.g., Ca²⁺ indicators having similar spectral responses, since the spectral response of each cell will be correlated with the unique optical signature of that cell.

In accordance with another aspect, membrane potential is monitored as an indicator of transmembrane ion fluxes in response to the interaction of a ligand target with the ligand (ligand), where the fluxes carry sufficient charge to change the electrochemical potential across a membrane. In certain embodiments, membrane potential in cells and organelles (e.g., mitochondria) is measured using potentiometric optical probes as described in The Handbook of Fluorescent Probes and Indicators, 9^{th} Ed: Chapter 23, (Molecular Probes, Invitrogen; available online as “The Handbook, Web Edition” at http://www.probes.com/handbook) and in Celis, Ed., Cell Biology: A Laboratory Handbook, 2nd Ed., Vol. 3, pp. 375-379 (1998) In accordance with this aspect, potentiometric optical probes are used to detect changes in membrane potential in response to the interaction of a ligand target with a ligand. Increases and decreases in membrane potential, or membrane hyperpolarization and depolarization, respectively, play a central role in cellular responses involved in, e.g., nerve-impulse propagation, muscle contraction, cell signaling and ion-channel gating. Potentiometric probes are important tools for studying these cellular processes, and for assessing cell viability, for high-throughput screening for new ligands. Potentiometric probes include, but are not limited to, the cationic or zwitterionic styryl dyes, the cationic carbocyanines and rhodamines, the anionic oxonols and hybrid oxonols, merocyanine 540, and JC-1. It is understood that one of skill in the art can select the dye for use in a particular embodiment, based on factors such as accumulation in cells, response mechanism and toxicity. In conjunction with imaging techniques provided herein, these probes can be employed to map variations in membrane potential across excitable cells with high levels of sampling frequency and spatial resolution.

In accordance with yet another aspect, the intracellular concentration of any one of various other cations, e.g., Na⁺ or K⁺ or anions, e.g., C1⁻, phosphate, pyrophosphate, nitrate, or sulfate, as an indicator of cellular responses to the interaction of the ligand target with the ligand (ligand). In certain embodiments, these ion concentrations are measured using indicators as described in The Handbook of Fluorescent Probes and Indicators, 9^{th} Ed., Chapter 22, (Molecular Probes, Invitrogen; available online as “The Handbook, Web Edition” at http://www.probes.com/handbook). Suitable cation indicators include, but are not limited to, benzofuranyl fluorophores linked to a crown ether chelator, e.g., PBFI and SBFI available from Invitrogen (Carlsbad Calif.), where cation selectivity is conferred by the cavity size of the crown ether. In certain embodiments, when a cation binds to SBFI or PBFI, the indicator's fluorescence quantum yield increases, its excitation peak narrows and its excitation maximum shifts to shorter wavelengths, causing a significant change in the ratio of fluorescence intensities excited at 340/380 nm. Suitable chloride (C1⁻) indicators include, but are not limited to, 6-methoxy-N-(3-sulfopropyl)quinolinium (SPQ), N-(ethoxycarbonylmethyl)-6-methoxyquinolinium bromide (MQAE), 6-methoxy-N-ethylquinolinium iodide (MEQ) or lucigenin, all available from Invitrogen (Carlsbad Calif.). Monochlorobimane is a fluorochrome that can be used as a glutathione probe.

3. Combining Color-Coded Cell Populations into a Mixed Cell Suspension.

As provided in the present invention, color-coded cell populations are combined to provide a mixed cell suspension that contains cells from a plurality of resolvable color-coded populations. Generally, cell populations are stained as described above, and each color-coded cell population is separated from the staining solution and transferred to a suspension buffer before combining. In one exemplary embodiment, the color-coded cells are spun down by gentle centrifugation and resuspended in a suitable buffer. Suitable buffers include hybridoma medium available from numerous sources (e.g., Sigma-Aldrich), assay buffers, or other defined media that can be selected by one of skill in the art depending on, e.g., the choice of host cell and/or the measurements being taken in a particular embodiment. Cell suspensions containing distinct color-coded cell populations can then be combined as desired to provide a mixed cell suspension. In the mixed cell suspension, cells are present from a plurality of resolvable color-coded populations, each with a distinct optical signature. Based on the distinct color-coded optical signature provided for each cell population, the origin of each cell in the mixed cell suspension is detectable when the fluorochrome-labelled cell is excited by laser sources and the emissions are collected through optical filters. It is understood that there is no known limit on the number of cell populations that can be combined, as long as each cell population can be resolved. If desired, cells can be maintained in suspension by placing cell-containing vessels on a shaker or nutator.

4. Analyzing Populations by Flow Cytometry.

In accordance with another aspect of the invention, the mixed cell suspension is contacted with a ligand and analyzed by a flow cytometry system. As described above, the mixed cell suspension introduced into a flow cytometry system contains color-coded cells that express ligand targets and have one or more indicator dyes to monitor cellular responses. During analysis by flow cytometry, the source population of each cell is determined by measuring color-coding fluorochromes, and the cellular response of the cell to the ligand is determined by measurement of the indicator dye.

In certain embodiments, the mixed cell suspension is contacted with a ligand by means of an automated mixing system, and analyzed by an automated sample input flow cytometry system. It is understood that the flow cytometry system must have low mix delay, wherein the cells in the suspension must be mixed rapidly and thoroughly with the ligand, so that all cells are exposed to the ligand within an acceptably short period of time, e.g., within about 0.5 seconds, or about 1 second, or about 1.5 seconds, or about 2 seconds, or about 2.5 seconds, or about 3 seconds. In one embodiment, the cells and the ligand must be mixed so that all cells are exposed to the ligand with a mix delay of about 2 seconds or less.

After the cells and the ligand are thoroughly mixed, the mixture is then injected into the flow cytometry system. “Injection delay” refers to the time period from mixing to injection. “Injection period” refers to the length of time necessary to inject the mixture of cells and ligand. It is understood that the optimal injection delay and injection period can be determined by one of skill in the art, depending on the conditions of a particular embodiment. For example, when the kinetics of the cellular response to interaction between a ligand target and a ligand are being determined, then the optimal injection delay and injection period must be controlled to permit measurement of the cellular response at a precise time. Similarly, when a short-lived transient cellular response is being measured, then the injection delay and injection period should be as brief as possible, to facilitate accurate measurement of the transient event. In one embodiment, the mixture is injected into the flow cytometry system within about 2 seconds (injection delay), and the cells are injected over a period of about 5 seconds (injection period).

In accordance with one aspect of the invention, cells and ligand are mixed and injected using an automated sample mixing and injection method and apparatus as described in detail in the following section entitled “Direct mixing and injection for high throughput fluidic systems” and in the co-pending patent applications entitled “Direct sample mixing and injection for high throughput fluidic systems” and “Sample analysis system employing direct sample mixing and injection” filed May 7, 2004, the entire contents of which are hereby incorporated by reference.

Analysis: Resolving Color Coded Cells in a Mixed Cell Suspension

In accordance with another aspect of the invention, one or more laser sources in the flow cytometry system is used to excite each fluorochrome-labelled cell in the mixture, after which fluorescence emissions are collected via the emissions collection pathway (fluorescence axis) of the instrument, and a specific region of each fluorochrome's emissions is transmitted to the detector which registers the wavelength(s) that are received. Fluorescence emitted by the fluorochrome(s) may be transmitted to the detector using, e.g., spectral steering mirrors, dichroics and filters. In certain embodiments, fluorescence emitted by the fluorochrome(s) may pass through a filter before it travels to a dichroic mirror which permits certain wavelengths of light to pass through while reflecting other wavelengths. Photodetectors and/or fluorescence detectors suitable for use in the present invention may be photomultiplier tubes or similar devices known in the art, which convert light signals into electrical impulses so that the light thereby detected may be associated with the color-coded cells passing through the flow cytometry system. Electrical signals from the photodetectors and fluorescence detectors are typically transmitted to an analysis system for purposes of display, storage or further processing so that one or more characteristics of the cells under analysis may be determined. Exemplary embodiments provided below describe the above-mentioned processes in greater detail.

As provided herein, the “signature” excitation/emission profile (optical signature) of each fluorochrome or combination of fluorochromes used to color-code cells, provides the ability to identify each color-coded cell. The optical signature of each color-coded cell allows it to be resolved from every other color-coded cell in the mixture. It is understood that is not necessary that each fluorochrome be excited optimally, nor that the emissions for each be collected at their optima, as long as the optical signature of each fluorochrome or combination of fluorochromes can be detected.

As noted above, there is no theoretical limit to the number of different cell populations, each with a unique optical signature, that can be mixed and analyzed as provided herein. It is understood that the only limit to the number of cell populations that can be resolved as provided herein, would arise from constraints such as the optical capacity of the flow cytometry system used in an embodiment, or the fluorochromes available for a particular embodiment. Cells having fluorochromes with similar, but not identical, emissions profiles can be combined in a mixed cell suspension, and emissions data from these fluorochromes can be combined into a single dual emissions plot (bivariate), and each cell in the mixed cell suspension can nonetheless be identified and resolved. In various embodiments, cells from 2, 3, 4, 5, 6, 7, 7, 8, 9, 10, 11, 12, 13, 14, 15, 16, 17 18, 19, 20, 21, 22, 23, 24, 25, 26, 27, 28, 29, 30, 35, 40, 45, 50, or more distinct cell populations were combined in a mixed cell suspension, and each cell was resolved from each other cell based on the profile of emissions (the optical signature) collected from each cell.

Analysis: Multiplexed Measurements of Cellular Responses in Single Color-Coded Cells.

The present disclosure provides a multiplexed design in which a flow cytometry system is used to make multiparametric measurements of several cellular properties of the same cell, and multiparametric measurements of a plurality of distinct cells, at the same or nearly the same time. The multiplexed system provided herein measures at least two aspects of each cell: cellular responses, e.g., calcium mobilization or alteration in transmembrane potential, triggered by the interaction of a ligand (ligand) with the ligand target expressed in a cell; and the excitation/emission profile that provides the optical signature to identify and resolve the source population of the cell whose cellular responses were measured.

In accordance with one aspect, the multiplexed multitarget system provided herein can be used to measure the interaction of a single ligand with multiple ligand targets. In certain embodiments, the multiplexed multitarget system provided herein is used to measure the interaction of a single ligand with multiple ligand targets in the same sample, e.g., the cellular responses and optical signature are measured for each cell in a sample that contains cells from a plurality of source populations, wherein cells from each source population express one or more distinct ligand targets. In certain embodiments, the system is used to identify and measure the cellular response(s) of each cell in a mixed cell suspension that contains cells expressing wild type ligand targets and cells expressing a plurality of variant or mutant ligand targets.

In accordance with another aspect, the multiplexed multitarget system provided herein can be used to measure the interaction of a single ligand target with multiple ligands. Generally, this requires that the interaction of the single ligand target with multiple ligands be carried out using a plurality of discrete samples containing cells expressing the ligand target, wherein each sample has been exposed to a different ligand. As provided herein, data obtained from discrete samples can be combined and compared for purposes of analysis of the interactions between ligand targets and ligands. Further as provided herein, data obtained using the present multiplexed multitarget system, can be used to analyze the interaction between multiple ligand targets and multiple ligands.

The following non-limiting exemplary embodiment demonstrates the multifunctionality of the present multiplexed multitarget screening method. In this non-limiting example, ten (10) distinct color-coded cell populations are prepared as described above, wherein the cells of each population express one wild type drug receptor and have a distinct optical signature. In this embodiment, the wild type drug receptor expressed by cells of one population is distinct from the wild type drug receptor expressed by each of the other 9 color-coded cell populations. A mixed cell suspension is prepared as described above by taking samples from each of the 10 distinct color-coded populations, the cells are loaded with a Ca²⁺ᵢ indicator dye, and the suspension is divided into 11 aliquots. Ten (10) aliquots are mixed with 10 distinct ligands to form 10 distinct assays. Each aliquot (assay) is injected into a flow cytometry system, and the optical signature and Ca²⁺ᵢ of each cell in the aliquot is measured. The signals obtained from each cell in the aliquot are deconvoluted and resolved. The remaining aliquot of the mixed cell suspension is not exposed to a ligand, but is injected into the flow cytometry system to provide quality control and baseline measurements of Ca²⁺ᵢ and optical signatures of the cells found in the mixed cell suspension. The optical signature of each cell signified what ligand target was expressed on that cell. The Ca²⁺ᵢ measurement of each cell provided a quantitative measurement of one cellular response triggered by the interaction of the ligand target expressed by that cell, with the ligand to which the cell had been exposed. Within an aliquot (assay), a comparison of the Ca²⁺ᵢ values obtained from cells obtained from each of the 10 distinct source populations provided a measure of the effect of a single ligand on different targets. A comparison of the Ca²⁺ᵢ values obtained from cells derived from the same source population in each of the 10 aliquots (the 10 different assays) provided a measure of the effect of different ligands on the same ligand target.

As noted above, the term “multiplexed” is intended to refer to simultaneous or near-simultaneous measurement of a plurality of signals. It is understood that the time frame for “simultaneous or near-simultaneous measurement” of signals means the time frame necessary to collect measures of cellular response(s) and optical signature from each cell in a sample containing a plurality of cells. It is understood that one of skill in the art can determine the experimental conditions that permit multiplexed multitarget screening as provided herein, depending on the ligand targets and the ligands of interest in a particular embodiment.

5. Resolving Populations and Deconvoluting Data

In accordance with another aspect of the invention, data from each cell in a sample are collected in a suitable computer-readable format, and are analyzed. It is understood that flow cytometry systems suitable for use in the present invention include data collection means, data analysis means, and recording means, e.g., a computer, wherein multiple data channels can record signals (data) emitted by each cell as it passes through a sensing region of the flow cytometer, and can analyze the data to derive the desired information. Signals or data emitted by each cell can be captured by means suitable for a particular embodiment, e.g., as an electrical signal, as an image that captures features of the cell (as described in U.S. Pat. No. 6,248,590), or as light captured by a charge-coupled device (CCD) detector. One of skill in the art can select a data capture means that is suitable for the signals generated in a particular embodiment and suitable for the data analysis

Data collected from each cell is used identify the source cell population of the cell, in order to resolve the distinct cell populations and their cellular responses. Generally, the cell populations are resolved from one another using logical gating analysis software employ suitable Boolean logic using AND, OR, NOT and other operators to resolve information relating to color-coded cells and cellular responses to test compounds.

The parameters to be measured include, but are not limited to, morphology, fluorescence, fluorescence polarization, fluorescence lifetime, incident light scatter, electromagnetic field induction, light absorbance, luminiscence, fluorescence resonance energy transfer (FRET), and bioluminescent resonance energy transfer BRET). In certain embodiments, multiple emissions or parameters are measured, and a dual parameter plot is used to more clearly resolve the difference between the results from “control” cell populations and results from various cell populations exposed to the drug compound(s) being tested. In an exemplary embodiment, multiple indicators of apoptosis are measured, in order to detect cells in various stages of apoptosis by distinguishing live cells, early apoptotic cells, dead cells, and debris in a mixed cell suspension exposed to a particular ligand. In one embodiment, nucleic acid stains and mitochondrial stains are used in a dual staining technique to detect apoptosis in living cells. (Poot et al., (1997) Cytometry 27:358-364; Hamori et al., (1980) Cytometry 1:132-135. (1980); Poot et al., Human Genetics 104, 10-14). In a particular embodiment, Hoechst 33342 is used as a DNA-specific (nucleus-specific) viable stain to indicate live cells, propidium iodide (PI) is used as a membrane-impermeant DNA stain that will only stain dead cells, and various SYTO™ stains that can stain nucleic acid (DNA and RNA) in the nucleus, mitochondria, and cytoplasm of live cells (Hoechst 33342, PI, and SYTO™ are available from Molecular Probes, Invitrogen). In this embodiment, live cells are positive for Hoechst 33342 staining and negative for PI staining, early apoptotic cells are Hoechst 33342 positive, PI negative, and show decreased (low) levels of SYTO staining, and dead cells are positive for PI staining. As provided herein, cells in a mixed cell suspension can be followed through various stages of apoptosis and decay. Another embodiment includes measurement of mitochondrial permeability transition, which is an early indicator of the initiation of cellular apoptosis. Mitochondrial permeability transition, typically defined as a collapse in the electrochemical gradient across the mitochondrial membrane, is measured by the change in the membrane potential, e.g., using a fluorescent cationic dye, 5,5′,6,6′-tetrachloro-1,1′,3,3′-tetraethyl-benzamidazolocarbocyanin iodide, commonly known as JC-1 (Cell Technology Inc., Mountain View Calif.). In another embodiment, cells in a mixed cells suspension are exposed to with a combination of Hoechst 33342 and JC-1, wherein Hoechst 33342 staining is used to assess chromatin fragmentation during apoptosis and JC-1 is used to detect cells undergoing mitochondrial permeability transition. It is understood that one of skill can design other embodiments that measure multiple parameters associated with a cellular response of interest in a particular embodiment.

In certain embodiments, data are collected in Flow Cytometry Standard (FCS) file format, which allows data to be replayed in real time. It is understood that many commercially available flow cytometers save data collected from cells into various release versions of FCS file format including FCS1.0, FCS2.0, or FCS3.0 file format. The FCS2.0 format was developed by the Data File Standard Committee of the Society for Flow Cytometry, and the standard has been published in a paper entitled “Data File Standard for Flow Cytometry” (1990), Cytometry 11:323-332. Information regarding FCS data file formats, is publicly available at the following site: http://nucleus.immunol.washington.edu/ISAC/Ref Data/refs.htm. It is further understood that non-FCS file structure is used is certain flow cytometry systems, e.g., in the EPICS and Profile flow systems (Beckman Coulter, Fullerton Calif.), wherein EPICS and Profile files can be converted to FCS files with the “FCAP-inp” communication program.

Once discrete populations have been identified by color-coding, data analysis of each population is performed by any one of several statistical methods. For example, if Ca²⁺ᵢ responses are measured in samples using Indo-1 as the indicator, the basal state of each population is defined by carrying out analysis on a diluent buffer control sample of the population. The mean relative Ca²⁺ᵢ level is calculated for each population, and one or more standard deviations are calculated. It is understood that the decision to use 1, 2, 3 or more standard deviations above the mean value is an empirical decision to select a “positive response threshold” value. For each color-coded population, the percentage of cells with values above the pre-selected positive response threshold after exposure to a ligand is calculated as a “response percentage.” The values of calculated response percentages are captured on a computer-readable medium; response percentages can be exported as ASCII files to a database, or can be converted into a suitable display such as a bar chart of the responses of each population to each ligand. A statistical measure of the difference between any one population exposed to ligand, and its control, can be performed using any suitable standard statistical test, e.g., a two state Chi Square test. In one embodiment, the Chi Square statistic then indicates the confidence level at which the ligand-treated sample triggers a cellular response that is different from the control. In another embodiment, Ca²⁺ᵢ response data is analyzed using an approach wherein the mean Ca²⁺ᵢ level and coefficient of variation is calculated for each population exposed to a ligand, and these values are compared with the corresponding values of the baseline control sample. Data analysis for other response parameters such as membrane potential or immunofluorescence may employ other methods, e.g., “percent positive” calculation, or determinations of population mean intensity. Yet other response measurements such as the use of JC-1 to evaluate mitochondrial membrane potential as described above, may involve more complex procedures such as dual emission plots to assess differences between control and treated populations.

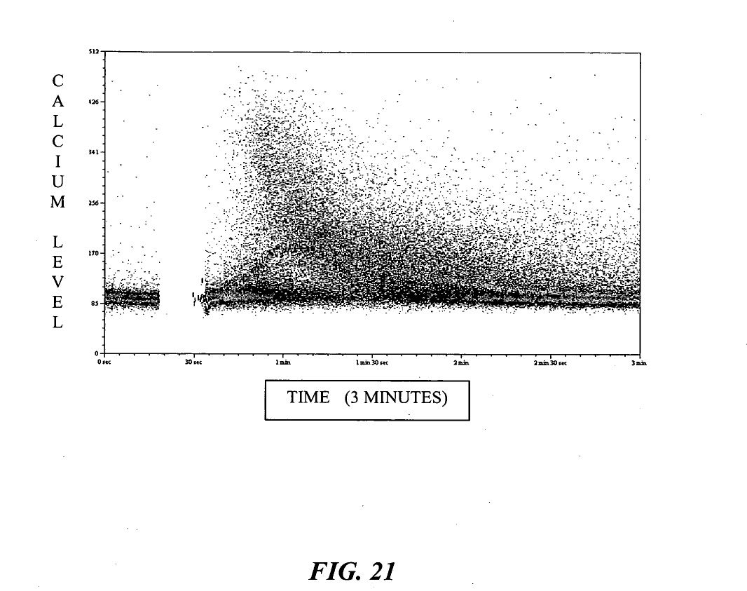

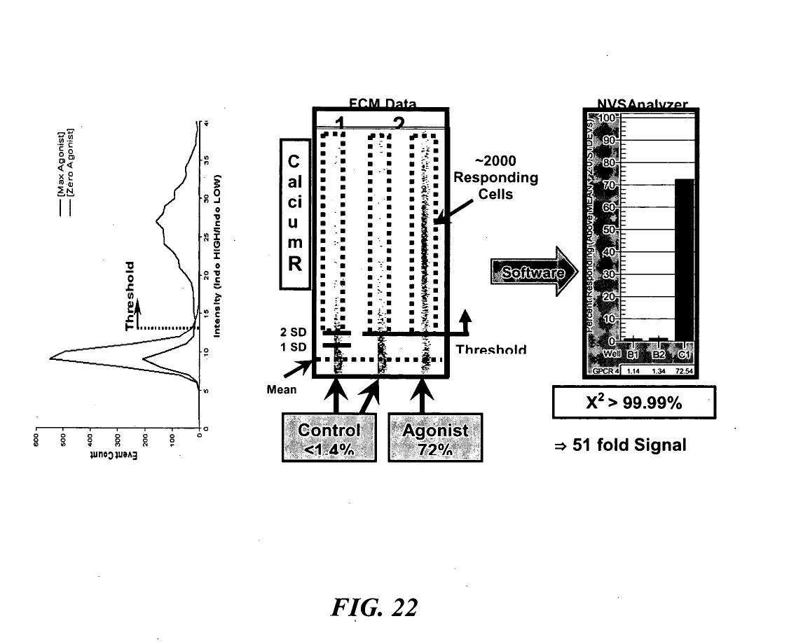

When measuring Ca²⁺ᵢ responses, the power of single cell analysis provides enhanced resolving power in terms of the ability to detect weakly active compounds. This enhanced resolution is revealed only when the data is analyzed as the percentage of cells in a population that exceeds a defined response threshold. FIG. 21 shows the distribution of Ca²⁺ᵢ levels measured in HEK293 cells transfected with the melanocortin 4 receptor (MC4R), as measured by flow cytometry, at rest (first 20 seconds) and after addition (following the gap) of 1 uM NDP-aMSH, a ligand for the MC4R. In FIG. 21, each dot represents a single cell, where more than one cell per pixel space is represented by transitions from darker to lighter shading in a black-and-white plot, and by transitions from red to yellow to white coloring in a color plot (these transitions are also captured quantitatively). The plot in FIG. 21 shows the results of continuously sampling the population. In FIG. 21, the solid line represents the average of the sampled population at each bin in the x-axis (time) as calculated from the single cell data. An instrument that measures the average response of the population over time, e.g., a spectrofluorimeter or a fluorescent imaging plate reader (FLIPR™), produces a plot like that of the solid line, whereas single cell analysis as provided herein yields different information. The average response measured by, e.g., a spectrofluorimeter or a FLIPR™ implies that the cellular response in all the cells in a sample develops relatively slowly over time, eventually reaching a peak value that is followed by a slow decline to the basal level. In contrast, single cell analysis of a sample provides the following results: (1) all the cells in a sample exhibit very similar low Ca²⁺ᵢ levels in the resting state; (2) only a subset of the cells respond to the ligand; and (3) the response is nearly instantaneous, so that the responding cells reach their peak very quickly. In addition, the peak is followed by a sustained period where a fraction of the cells can be detected as having Ca²⁺ᵢ levels that are still elevated above the basal state; this result is hardly apparent in the “average response” plot. This behavior is consistently seen for all GPCRs analyzed at the single cell level as provided herein. A rotated histogram plot of such a response, and of the basal population, is shown in the left panel of FIG. 22, where the Ca²⁺ᵢ level is shown on the x-axis and the number of cells attaining each Ca²⁺ᵢ level is on the y-axis. Because the response exhibits such rapid onset ‘all or none’ behavior, the response can be quantified by establishing a threshold Ca²⁺ᵢ level and determining the percentage of the sample that crosses the threshold level, or the “percent responding.” All samples exposed to test compound can then be compared to the percentage of cells exceeding the threshold Ca²⁺ᵢ level while in the basal state in the presence of a buffer control sample. The threshold is set by calculating the mean of the Ca²⁺ᵢ level in the buffer control sample and calculating one (1) or more standard deviations above the mean. The choice to use one or more standard deviations is based on minimizing the error between buffer control samples and fully activated positive control samples. Typically one to three standard deviations is used as the threshold error range. The center panel of FIG. 22 shows a response plot of two buffer control samples and an experimental sample containing 5HT2A receptor-bearing cells stimulated with 1 uM serotonin (5HT). Results using autosampling flow cytometry and single cell analysis show that the Ca²⁺ᵢ levels for the three individual ‘plugs’ are shown from left to right. A threshold level at two standard deviations above the mean Ca²⁺ᵢ level is shown by the arrow. Because the single cell flow cytometry as provided herein can analyze samples rapidly, about 100 to 200 cells, or more, can be analyzed in a short period. Because the data is reduced to a binomial format, where the cells either cross the threshold or do not, the results are correctly analyzed by a Chi Square technique using one degree of freedom calculated as: (percent observed−percent expected)²/(percent expected). Data analysis is implemented in an automated software algorithm that automatically calculates Chi Square values along with the response frequency for each sample. The percentage of cells that are expected to cross the threshold (percent expected) is determined from the buffer control sample, and the percentage of cells observed to cross the threshold (percent observed) is the frequency that is actually measured. Response frequencies can be represented several ways, and an example is shown in the right panel of FIG. 22 where the frequencies are a simple bar chart on a per sample basis. In this example showing results for a ligand-stimulated sample, the signal to baseline ratio (stimulated to unstimulated response frequency) is over 50. The large number of cells analyzed, the large confidence levels determined by Chi Square analysis, and the high signal to baseline ratio derived from this analysis algorithm, are the bases for the increased resolving power and high statistical significance of the analysis provided herein.

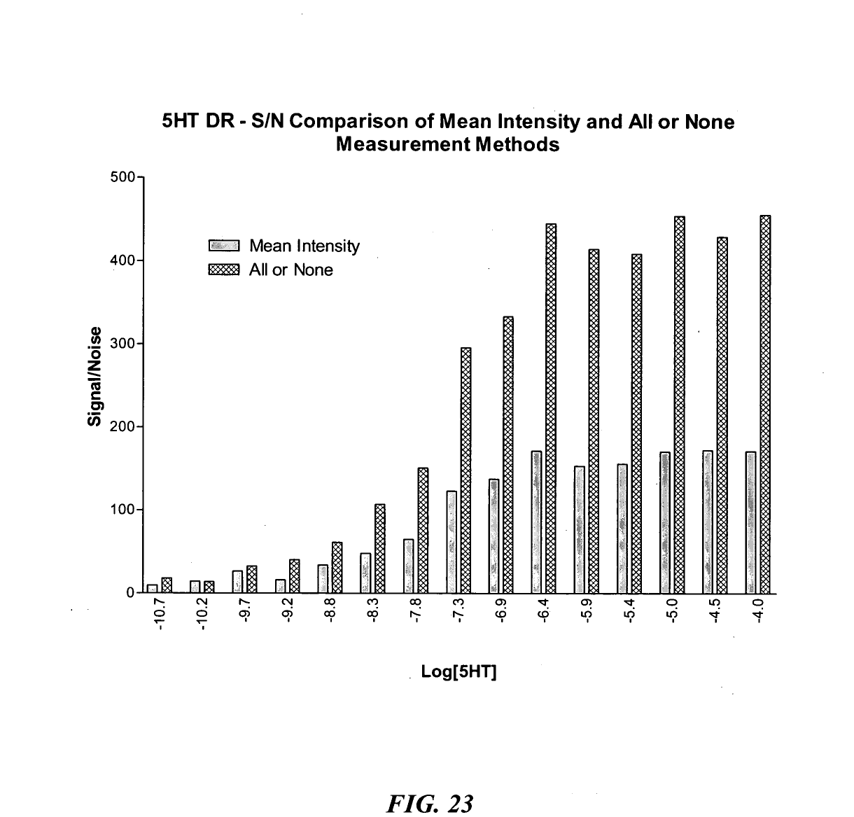

In one non-limiting example of the value of the high resolution afforded by the response analysis algorithm, illustrated in FIG. 23, 5HT2A receptor-bearing cells were stimulated with increasing concentrations of the cognate ligand serotonin (5HT) using automated flow cytometry. Data from the samples were analyzed to determine either the average response magnitude or the frequency of responding cells, and the signal to noise value was calculated using the formula (stimulated value−baseline control value)/(baseline value). FIG. 23 shows that at all concentrations, the signal to noise of the frequency analysis calculation exceeded that of the sample average calculation. More importantly, FIG. 23 shows that the signal to noise of the frequency analysis calculation exceeded that of the sample average calculation at diminishing concentrations of ligand. This result indicates that the frequency analysis method provided herein allows resolution of weakly active ligands that would not be detected by a sample average calculation method.

In one non-limiting example described in Example 3, a mixed cell suspension containing eight distinct color-coded populations of human HEK293 cells was analyzed by single cell flow cytometry, and the eight distinct cell populations were resolved simultaneously as shown in FIG. 19. By way of illustration, the population of cells stained with Alexa 594, indicated in FIG. 19.C., was isolated in the data analysis software by building informatic “regions” around each of the other populations as identified in the other three bivariate dot-plots presented in FIGS. 19.A., 19.B., and 19.D., and “removing” those other populations from the display in FIG. 19.C. When the results are displayed in a bivariate (2-D) plot, this approach to data and signal processing leaves only the signals corresponding to the expected signal from the Alexa 594-labelled cell population. This approach facilitates the analysis of cellular responses of each distinct cell population, since the signals from other cell populations have been removed. Once the populations are clearly resolved, analyses of the cellular responses (e.g., Ca²⁺ᵢ mobilization or changes in transmembrane potential) is performed for each population separately and the results are recorded.

Optional Recovery Step

The present disclosure further provides an optional sorting step to recover cells analyzed by the process described herein. Data accruing from the flow cytometric measurements described above can be analyzed rapidly enough that electronic cell-sorting procedures could be used to sort and recover cells by fluorescence-activated cell sorting (FACS). Sorting, or recovery, of cells is not required for the present method because the labelled cells that are analyzed and resolved as described herein are taken from source cell populations that can be maintained independently. Accordingly, the system described above can be used to identify cells having desired qualities, and thereby identify the cell source populations having the desired qualities, while the cells that were actually analyzed by flow cytometry can be discarded (i.e., not recovered) because additional cells can be recovered from the corresponding source cell population.

In accordance with another aspect, sorting and recovery of individual cells that have been analyzed may be desirable. Examples of sorting from multiplexed populations based on response phenotypes include sorting from multiple populations of cells bearing transfected, up-regulated or endogenous receptors into two more sorting receptacles to improve assay properties of multiple assay candidates, and sorting from multiple populations of primary or immortalized cells using one or more parameters as measures of responsiveness or non-responsiveness to test compounds. Analysis and sorting of multiple populations allows further comparative molecular and biochemical analysis of the difference(s) between responsive and non-responsive cells.

Direct Sample Mixing and Injection for High Throughput Fluidic Systems

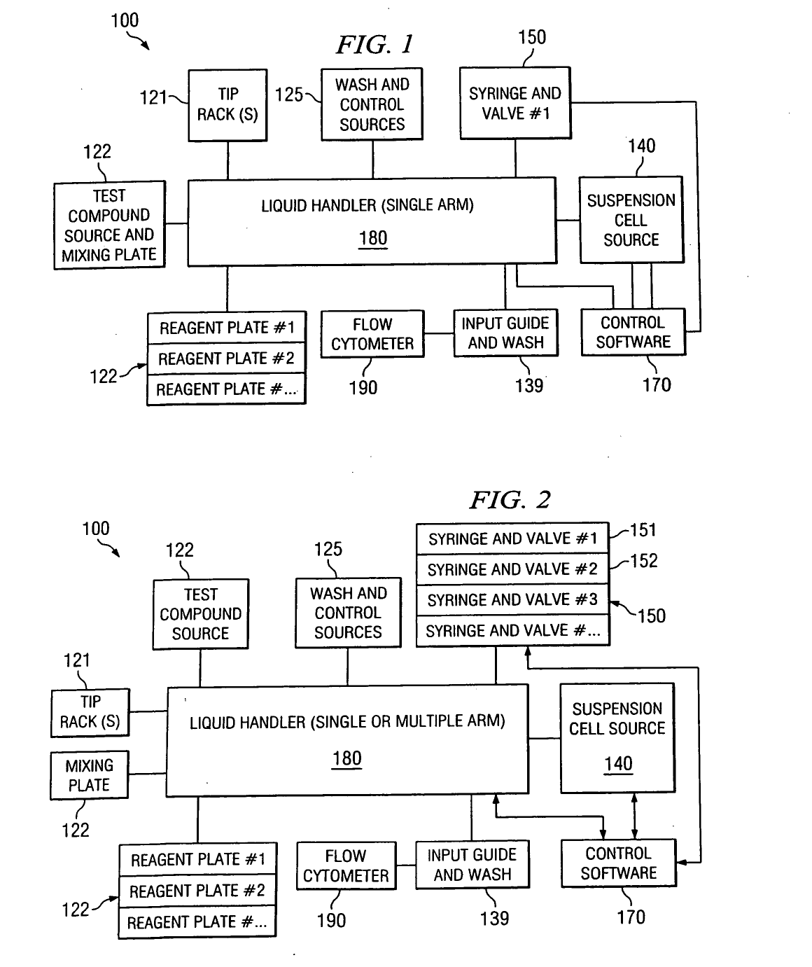

In one exemplary embodiment, the present invention is practiced using a system and method of mixing and injecting discrete sample mixtures into a flow cytometer or other sample analysis apparatus, as described below and illustrated in FIGS. 1-16. In accordance with some exemplary embodiments, for example, a sample injection guide may couple a liquid handling apparatus with a sample analysis apparatus, facilitating injection of discrete sample mixtures into a fluidic system of the apparatus.

As set forth in more detail below, a sample analysis system may generally comprise: a liquid handling apparatus operative to prepare a discrete sample mixture; a sample analysis apparatus; and an injection guide coupled to the analysis apparatus; the injection guide operative to receive the discrete sample mixture from the liquid handling apparatus and to provide the discrete sample mixture to a fluidic system of the analysis apparatus. In accordance with some embodiments, the injection guide may comprise: a guide well operative to engage a pipette tip manipulated by the liquid handling apparatus; and a port in fluid communication with the guide well and operative to receive the discrete sample mixture from the pipette tip and to communicate the discrete sample mixture to the fluidic system. The guide well and the port may be in continuous fluid communication with the fluidic system.

Turning now to the drawing figures, FIG. 1 is a simplified block diagram illustrating functional components of one embodiment of a sample analysis system incorporating elements of a direct sample injection system, and FIG. 2 is a simplified block diagram illustrating functional components of another embodiment of a sample analysis system incorporating elements of a direct sample injection system.

The functional description set forth below is primarily directed to operational characteristics of the FIG. 2 embodiment which may employ a dual pipetting arm liquid handler arrangement, though a single pipetting arm arrangement, such as illustrated in FIG. 1, may also have utility in various applications. Those of skill in the art will appreciate that a sample analysis system as contemplated herein may be susceptible of numerous alterations and modifications, and that the particular configuration of structural components may be selectively adjusted in accordance with myriad considerations including, but not limited to: overall system requirements; size or scale limitations of one or more structural elements; implementation, programming instructions, and computational bandwidth of various processing components; desired sample throughput rates; and other factors. In particular, the present disclosure is not intended to be limited by the number of articulated arms employed by any particular liquid handler apparatus.

As illustrated in FIGS. 1 and 2, an exemplary sample analysis system 100 generally comprises an analysis apparatus such as a flow cytometer 190, for example, and a liquid or sample handling and injection system, such as liquid handler 180. As contemplated herein, references to “direct sample injection” and similar terms are generally related to a process of delivering discrete sample mixtures from liquid handler 180 to an independent fluidic system such as may be incorporated or integrated in a sample analysis apparatus (e.g., flow cytometer 190); it will be appreciated that, in this context, the term “independent” generally refers to a fluidic system of a sample analysis apparatus that is distinct from, or not necessarily integrated with, the structure (in general) and the fluidic system (in particular) associated with liquid handler 180, though used in conjunction therewith in system 100.

In some embodiments, flow cytometer 190 may be implemented in fluorescence activated cell sorting (FACS) applications; additionally or alternatively, flow cytometer 190 may be employed in any of various sample analysis applications generally known in the art or developed and operative in accordance with known principles. In alternative implementations of system 100, flow cytometer 190 may be supplemented or replaced by any of various different types of sample analysis apparatus benefiting from direct sample injection functionality as set forth in more detail below. For example, one such alternative apparatus may include suitable structural elements allowing or enabling various microfluidic applications; those of skill in the art will appreciate that a direct sample injection system may have utility in numerous environments with minimal or no modification.

During use, liquid handler 180 may be operative (under microprocessor or computer control, for example) to prepare samples to be analyzed and to deliver sample material or other liquid mixtures to a flow cytometer 190 or another sample analysis apparatus through a sample injection guide component 139. In that regard, liquid handler 180 in the FIG. 2 arrangement may be embodied in or incorporate any of various commercially available, computer or microprocessor controlled, dual arm liquid handling stations such as, for example, a Cavro RSP 9000 unit; similarly, the FIG. 1 liquid handler 180 may be embodied in or comprise any single arm liquid handling station such as may be generally available or as may be developed and operative in accordance with the functional characteristics set forth herein.

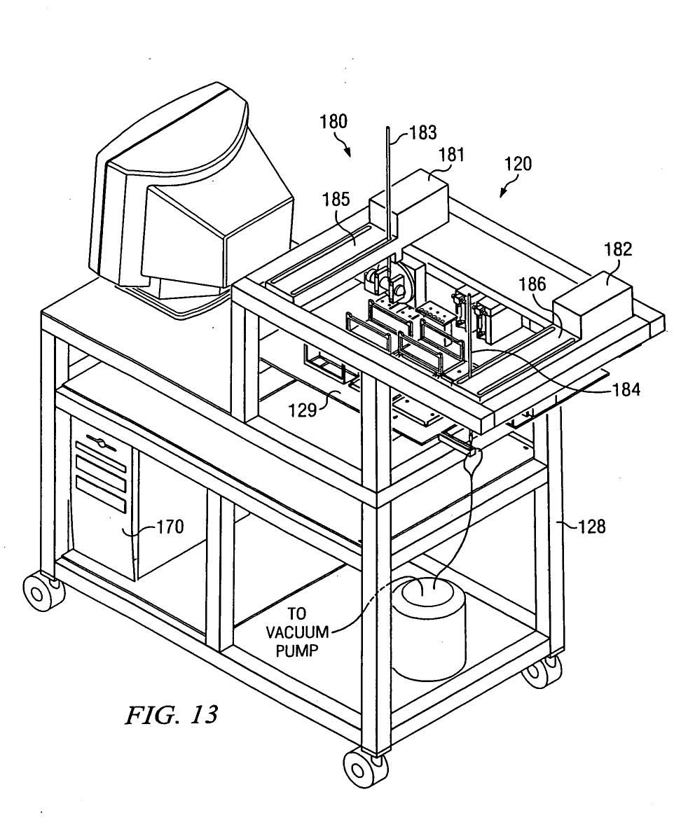

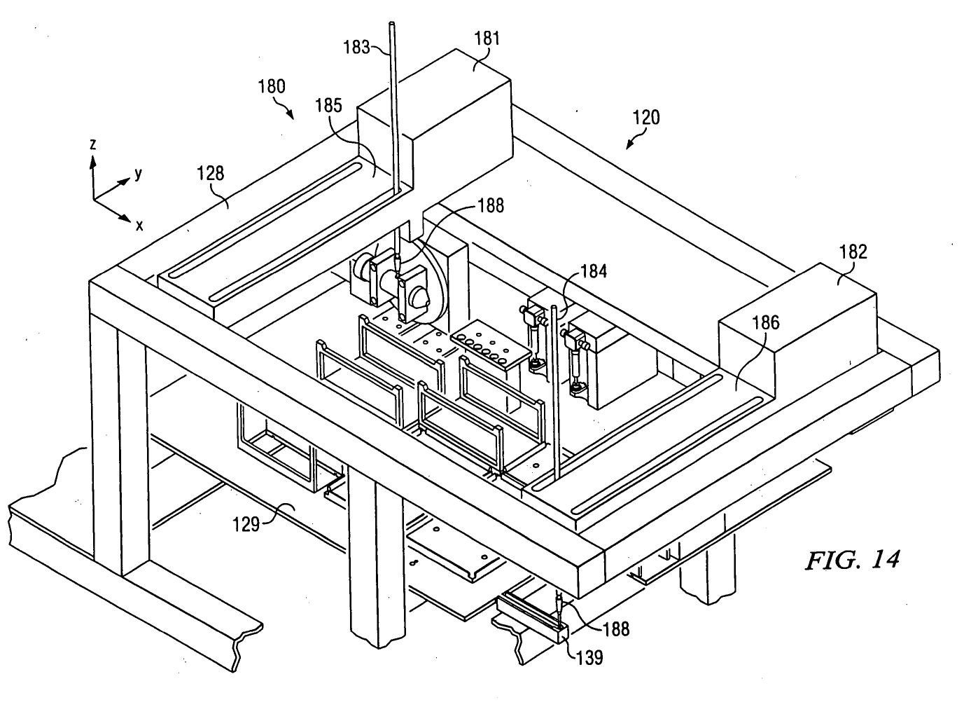

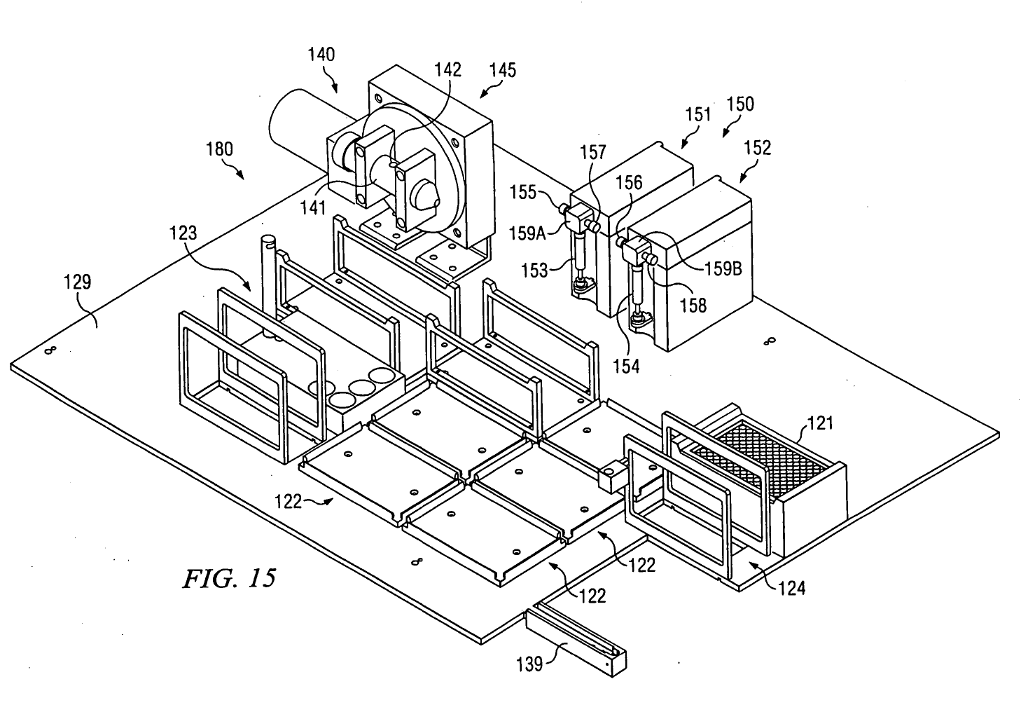

With reference now to FIGS. 13-15 in addition to FIGS. 1 and 2, it is noted that FIG. 13 is a simplified perspective diagram illustrating components of one embodiment of a sample analysis system incorporating a direct sample injection system, FIG. 14 is a simplified perspective diagram illustrating components of one embodiment of a direct sample injection system, and FIG. 15 is a simplified perspective diagram illustrating additional components of the direct sample injection system of FIG. 14.

Liquid handler 180 may generally be configured and operative to implement disposable pipette tips on any number of pipetting arms; as set forth above, while the exemplary embodiment of FIGS. 2, 13, and 14 employs two pipetting arms (reference numerals 181 and 182), systems incorporating one arm (FIG. 1), as well as systems incorporating more than two arms, are also contemplated. Such systems employing an arbitrary number of pipetting arms may be implemented in accordance with the principles and functional attributes described herein. In the exemplary system 100, a respective pipetting probe 183,184 may be suspended from a respective translational support structure 185,186 associated with each respective arm 181,182. Such pipetting arm assemblies accommodate rapid, precise movement of probes 183,184 in x, y, and z (i.e., Cartesian) coordinate directions. For many applications, translation in approximately 0.003 inch (0.076 mm) increments in a particular coordinate direction may readily be achieved using conventional automated or microprocessor controlled liquid handlers; such precision may be sufficient, but may not be necessary, for typical uses. It will be appreciated that the degree of precision with which a pipetting arm (181,182) and its associated support structure (185,186) and probe (183, 184) are moved may be a function of various factors; the present disclosure is not intended to be limited by parameters affecting accurate and precise placement of structural elements in traditional liquid handling systems.

Pipetting arm 181,182, structure 185,186, and probe 183,184 combinations are generally operative to manipulate probes 183,184 in three-dimensional space, enabling probes 183,184 selectively to engage a pipette tip (reference numeral 188 in FIG. 14) which may be fabricated of plastic, acrylic, latex, or other suitable materials as generally known in the art. In that regard, probe 183,184 may be lowered into a rack of pipette tips (reference numeral 121) for coupling of probe 183,184 with a cooperating pipette tip 188. Some such pipette tips 188 currently available may have, for example, a fluid volume capacity of about 20-1000 μl (e.g., Tecan Genesis tips, from VWR/Quality Scientific Products, are available in the foregoing capacity range, and may be suitable for various applications involving automated or semi-automated pipetting procedures).

In some embodiments, a coupling structure or component may facilitate coupling of probe 183,184 with a particular type of pipette tip 188 having known structural dimensions. Specifically, FIGS. 6, 7, and 8 are simplified diagrams illustrating perspective, side elevation, and axial views, respectively, of one embodiment of a coupling component allowing a pipette probe to engage a pipette tip. As illustrated in FIGS. 6-8, a coupling component 110 may generally comprise a conduit 112 through which fluid may be communicated. Coupling component 110 may be fabricated of plastic (such as DELRIN™ for example), acrylic, metal, or other material having suitable strength, rigidity, and corrosion resistance characteristics, for example, which may be application-specific.

Coupling component 110 may comprise an appropriate structural element configured and operative to secure coupling component 110 to probe 183,184; specifically, probe 183,184 and coupling component 110 may be sealingly engaged, preventing leakage or other liquid loss at the juncture therebetween. In the exemplary embodiment, structural coupling or interconnection between probe 183,184 and coupling component 110 is represented as effectuated at a threaded portion 111. It will be appreciated, however, that coupling of probe 183,184 and coupling component 110 may be achieved using other structural elements such as, for example, a quick-disconnect mechanism, a hose barb, or other coupling device having utility in fluidic systems.

Similarly, coupling component 110 may additionally comprise an appropriate structural element configured and operative to secure pipette tip 188 to coupling component 110; as with the connection set forth above, coupling component 110 and pipette tip 188 may be sealingly engaged, preventing leakage or other liquid loss at the juncture therebetween. In the exemplary embodiment, structural coupling or interconnection between coupling component 110 and pipette tip 188 is represented as effectuated at an angled portion 114 operative (e.g., like a hose barb) to engage, under pressure, a cooperating open end of pipette tip 188 having a correspondingly angled inside diameter dimension as generally known in the art. It will be appreciated that coupling of pipette tip 188 and coupling component 110 may be achieved using other structural elements having utility in fluidic systems. In some embodiments implementing automated liquid handling apparatus and techniques, coupling component 110 may additionally allow or enable automated ejection (i.e., disengagement or decoupling) of pipette tip 188 from angled portion 114.

During pipetting operations when coupling component 110 is interposed between probe 183,184 and pipette tip 188, liquid may be communicated from probe 183,184 into conduit 112, and vice-versa, at end 115; similarly, liquid may be communicated from conduit 112 to pipette tip 188, and vice-versa, at end 113. It will be appreciated that the various elements, in general, and the specific structural arrangement, in particular, of coupling component 110 may be susceptible of various modifications, and that aspects of the exemplary structure depicted in FIGS. 6-8 may be selectively dimensioned, altered, omitted, or rearranged in accordance with numerous considerations including, but not limited to, the dimensions and other structural characteristics of probes 183,184, pipette tip 188, or both. For example, where probes 183,184 and pipette tip 188 are suitably constructed for direct coupling or other unassisted engagement, it may be possible to omit coupling component 110 from the fluidic path (i.e., coupling component 110 may not be required for proper operation of some embodiments of liquid handler 180).

As illustrated in FIGS. 1 and 2, a sample analysis system 100 may generally comprise a pump system 150 configured and operative to control fluid flow and liquid handling procedures. As indicated in FIGS. 2 and 15, the pipetting function for each respective pipetting arm 181,182 and probe 183,184 assembly may be driven or otherwise influenced by a respective pump system 151,152. In the exemplary implementation, pump systems 151,152 may be embodied in or comprise computer or microprocessor controlled, servo motor driven syringe and diverter valve systems in fluid communication with the interior of probes 183,184 through flexible tubing, for example, or through some other suitable fluidic path or conduit. One exemplary apparatus, the Hamilton PSD3 Servo syringe pump, is commercially available and may be suitable for use in accordance with the present disclosure.

In operation, a syringe motor (not shown in FIG. 15) may receive commands from control software, firmware, or other programming instruction sets; in FIGS. 1, 2, and 13, such control functionality is represented generally by the reference numeral 170. Accordingly, the syringe motor may be instructed selectively to withdraw a syringe plunger (e.g., to load a syringe 153,154) or to advance the syringe plunger (e.g., to expel contents of syringe 153,154). In some systems, a diverter valve 159A,159B may also receive commands from control software or some other processing and control component 170 (i.e., hardware, firmware, or software). In that regard, diverter valve 159A,159B may be instructed selectively to allow communication of liquids between syringe 153,154 and a buffer supply source (reference numeral 125 in FIGS. 1 and 2), for example, through a port 155,156, or between syringe 153,154 and probes 183,184 through an alternative port 157,158.

The foregoing arrangement allows syringes 153,154 to fill with an appropriate buffer material (such as PBS or HBSS, for instance) or with other chemical or biological reagents, and selectively to drive the fluid contents of syringes 153,154 through the interior (conduit 112) of coupling component 110 and into or through pipette tip 188 as set forth in more detail below. In particular, the volume of material drawn into or dispensed from pipette tip 188 coupled to a respective probe 183,184 may be controlled (e.g., under hydraulic control) by selective operation of respective pump systems 151,152.

The foregoing operation and various other functional characteristics of system 100 may be controlled by processing component 170. In that regard, processing component 170 may be embodied in or comprise one or more computers, microprocessors or microcomputers, microcontrollers, programmable logic controllers, field programmable gate arrays, or other suitably configurable or programmable hardware components. In particular, processing component 170 may comprise hardware, firmware, software, or some combination thereof, configured, appropriately programmed, and operative selectively to control operational parameters or otherwise to influence functionality of components of system 100. It will be appreciated that processing component generally comprises a computer readable medium encoded with data and instructions, these data and instructions causing an apparatus (such as any of the various components of system 100, in general, and liquid handler 180, in particular) executing the instructions to perform some or all of the functionality set forth herein.

Parameters which may be affected or controlled by processing component 170 may include, but are not limited to, the following: timing of movement and precise three-dimensional positioning of arms 181,182, support structures 185,186, probes 183,184, and more particularly, some combination thereof; timing and precise control of pump systems 151,152 including syringes 153,154 and valve assemblies 159A,159B, influencing the volume of fluid in pipette tips 188 and the destination thereof, timing and characteristics of mixing operations (as set forth below); sample injection rates through guide 139 and to an independent fluidic system; and other factors.

Accordingly, processing component 170 may be capable of transmitting control signals or other instructions to various other electrical or electromechanical system elements; it will be appreciated that cooperating electrical and mechanical elements (such as motors, servos, actuators, racks and pinions, gearing mechanisms, and other interconnected or engaging dynamic parts, for example) have been generally omitted from the drawing figures for clarity, as have the various electrical connections and wiring therebetween. In that regard, those of skill in the art will appreciate that control signals may be transmitted from, and feedback from various electromechanical components may be received by, processing component 170 in accordance with any of various communication technologies and protocols having utility in interconnecting or otherwise coupling computer peripheral devices and other electronic components. Specifically, devices implemented in system 100 may be coupled to enable uni- or bi-directional data communication using serial or Ethernet connections, for example, or other standards such as Universal Serial Bus (USB) or Institute of Electrical and Electronics Engineers (IEEE) Standard 1394 (i.e., “FireWire”) connections, and the like. In some embodiments, such coupled components may employ wireless data communications techniques such as BLUETOOTH™ for example, or other forms of wireless communication technologies based upon infrared (1R) or radio frequency (RF) signals.

As indicated in FIGS. 13 and 14, an automated pipetting arm assembly 120 including liquid handler 180 may be mounted on a frame 128, allowing pipetting arm 181,182 and probe 183,184 assemblies to address several different stations (e.g., pipette tip rack station 121, a microwell plate station 122, a tube station 123, and a waste bag station 124) selectively positioned or disposed on a deck or platform 129 generally positioned below arms 181,182. Frame 128 and platform 129 may be constructed of metal (such as aluminum or steel, for example), plastic, acrylic, fiberglass, or other suitably rigid material capable of bearing weight of arms 181,182 and other components of liquid handler 180, pump systems 151,152, stations 121-124, and attendant hardware or consumables disposed thereon.

In particular, as noted above, platform 129 may support several selectable stations 121-124. Examples of the stations include, but are not limited to the following: a microwell plate station (such as indicated at 122) for test compounds (ligands); a microwell plate station (such as indicated at 122) for mixing the cells and test compounds (ligands) where wells may or may not contain dilution buffer or test compounds at the outset; a rack containing tubes (such as indicated at 123) for holding buffers, probes, or test compound standards; waste bag stations (such as indicated at 124) for discarding tips and for expelling priming buffer from probes 183,184; and racks (such as indicated at 121) for holding predispensed trays of pipette tips. It will be appreciated that various other types of stations accommodating different consumables or other items having utility in experimentation may also be included; further, the specific number and orientation of the various stations 121-124 may be altered in accordance with desired system capabilities or application requirements.

As indicated in FIG. 15, platform 129 may additionally support a sample injection guide 139. In that regard, FIGS. 9, 10, 11, and 12 are simplified diagrams illustrating perspective, plan, side elevation, and axial cross-section views, respectively, of one embodiment of a sample injection guide. In some embodiments, guide 139 may be rigidly or fixedly attached to platform 129 or to some other structural element of frame 128. The attachment may be substantially permanent, for example, such as may be achieved by welds, rivets, pressure or heat sensitive adhesives, or other substantially permanent attachment mechanism; alternatively, guide 139 may be removably attached to platform 129 or frame 128 such as by screws, bolts, tabs and slots, or other cooperating structural arrangements, for example. It will be appreciated that a removable or adjustable attachment mechanism may provide flexibility for various applications. In some alternative embodiments, guide 139 may be attached, coupled, incorporated, or otherwise integrated into the structure of flow cytometer 190 or other sample analysis apparatus. In such embodiments, it may be desirable to modify or otherwise to adjust the dimensions or relative positioning of platform 129, other components of frame 128, or some combination thereof, to allow engagement of pipette tip 188 with guide 139 as set forth in detail below.

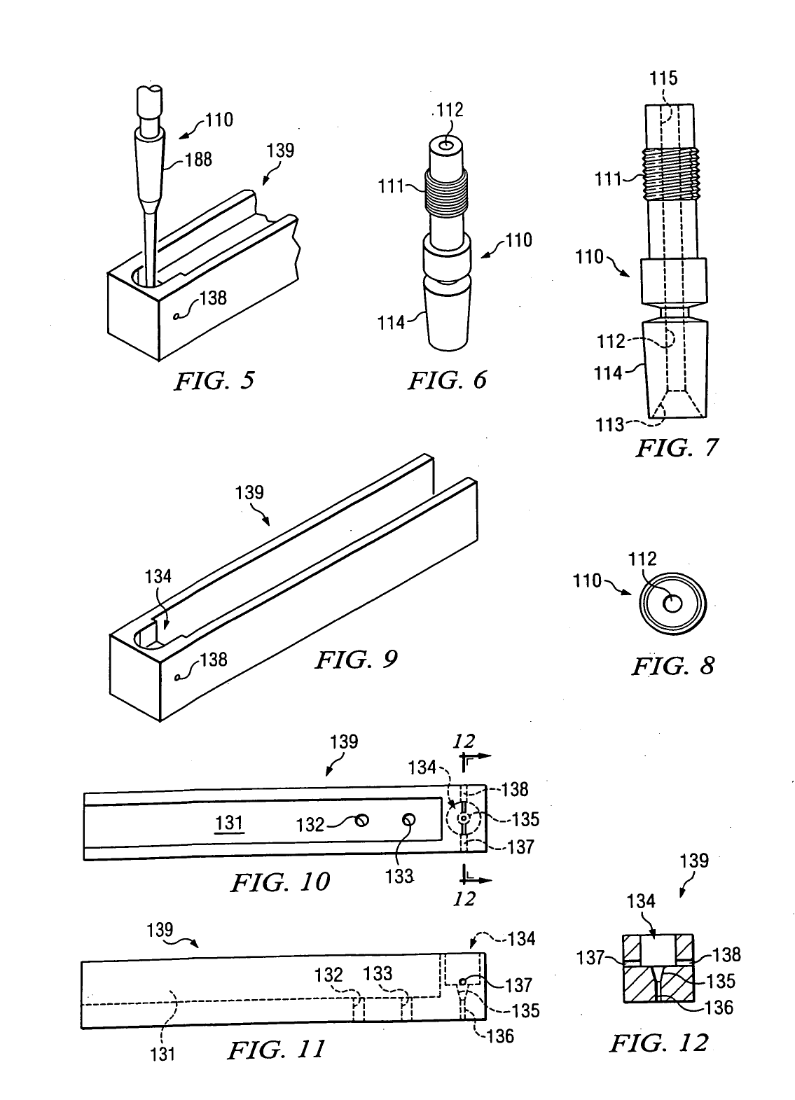

FIG. 5 is a simplified diagram illustrating a perspective view of one embodiment of a sample injection guide engaged with a pipette tip during use. Specifically, guide 139 may be constructed and operative to engage an end of pipette tip 188 and to communicate fluid from pipette tip 188 to the fluidic system of flow cytometer 190 or another sample analysis apparatus. A detailed description of one embodiment of guide 139, as well as some functional characteristics thereof, is provided below.

General Functionality

As set forth in detail above with specific reference to FIGS. 2 and 13-15, functional and mechanical drawings illustrate various components of one embodiment of a sample analysis system 100 employing a dual arm direct sample injection system; the functional attributes of a simpler, single arm embodiment (FIG. 1), as well as those of more complicated embodiments employing more than two pipetting arms, will be readily inferred from the following detailed description of operational characteristics.

Each respective arm 181,182, support structure 185,186, and probe 183,184 assembly may selectively visit tip rack 121 (or a selected, designated, or predetermined one of a plurality of tip racks 121, for example), seal a pipette tip 188 onto the end of each respective probe 183,184, and withdraw the sealed pipette tip 188 in preparation for movement to another station 122-124 on platform 129. As set forth above, probe 183,184 (either in conjunction with coupling component 110 or independently, for example) may form a sufficiently complete seal with pipette tip 188 to allow pipette tip 188 to be withdrawn from tip rack 121 without falling off when probe 183,184 is withdrawn. In particular, such a seal may also be sufficiently complete to prevent air or fluid leakage when fluids are moved into pipette tip 188 from either a reservoir or from a respective pump system 151,152—as described above with particular reference to FIG. 15, pump systems 151,152 may provide fluid (through probes 183,184) and drive volume aspiration and displacement for pipette tip 188.

Coupling component 110 may provide improved sealing between pipette tip 188 and probes 183,184. In one embodiment, for example, coupling component 110 may be fabricated of DELRINM plastic, though other plastics, acrylics, fiberglass, and other materials may also be suitable. Coupling component 110 may be constructed to precise dimensional specifications, and may generally be designed and operative to accommodate disposable pipette tips 188 from approximately 20 μl to approximately 1000 μl volume capacity. As set forth above with specific reference to FIGS. 6-8, different disposable pipette tip 188 products may require or substantially benefit from different specifications and structural composition of coupling component 110.

In operation, pipetting arm 182 may be used to inject successive discrete sample mixtures into flow cytometer 190 through guide 139. Initially, arm 182 may position probe 184 at a waste bag station 124, or at some other designated or selected waste vessel location; the attached pipette tip 188 may then be filled entirely (i.e., until a small excess amount is expelled as waste) with working liquid (e.g., buffer). In some embodiments, a desired buffer solution may be drawn through port 156 from a buffer reservoir (reference numeral 125 in FIGS. 1 and 2) into syringe 154. As set forth above, the selective connectivity of syringe 154 with buffer reservoir 125 or the pipette fluid path (via ports 156,158, respectively) may generally be controlled by valve 159B in line with syringe 154; accordingly, the contents of syringe 154 may then be provided to probe 184 and pipette tip 188 through port 158. Filling pipette tip 188 entirely with buffer may remove compressible air bubbles from pipette tip 188 and prevent a discrete sample mixture from being displaced back up into pipette tip 188 during later operations, for example, upon engagement of tip 188 with guide 139 when positive pressure from the fluidic system of flow cytometer 190 communicates with the contents of pipette tip 188. In some simplified dual arm liquid handling embodiments, arm 182 may be used strictly for retrieving discrete sample mixtures from selected locations on platform 129 and successively injecting these discrete sample mixtures into flow cytometer 190 or another analysis apparatus.

In coordinated or substantially simultaneous operations, pipetting arm 181 may also have buffer fluid within the tubing path (i.e., through probe 183 and to pipette tip 188). As described above with specific reference to arm 182, this fluid flow may be regulated through selective operation of syringe 153 and valve 159A of pump system 151. Such buffer fluid may facilitate reduction of compressible air in the tubing path of arm 181. In embodiments where probe 183 of arm 181 does not communicate with the high pressure fluidic system of a sample analysis apparatus (i.e., does not couple or engage pipette tip 188 with guide 139), the buffer solution may not be required to fill pipette tip 188. In the exemplary dual arm liquid handling embodiments, arm 181 may be employed to retrieve cell samples from a cell suspension system (described below) and to dispense these samples into an assay or microwell plate at a selected station 122 on platform 129, to retrieve test compounds (ligands) or buffer solution from one or more additional stations 122 at predetermined locations on platform 129 and to dispense same into an assay or microwell plate at a specific station 122 on platform 129, and to perform mixing functions (e.g., mixing the cell samples with compounds, mixing compounds with diluting reagents, or both).

Timing of movements for arm 181 may be keyed off the priorities and movements of arm 182. Specifically, to prevent collisions between arms 181,182, movement conflicts may be resolved, for example, by providing priority to arm 182; in such an embodiment, arm 181 may be required to wait for arm 182 to complete high priority tasks before arm 181 progresses to its next step or location in space. More complicated dynamic prioritization strategies may be employed in sophisticated liquid handling techniques. In the exemplary embodiment employing a strategy in which arm 182 has permanent priority, arms 181,182 may be synchronized to coordinate motions for maximal movement efficiency. It will be appreciated that the particular synchronization strategy employed may be application specific, and accordingly may be affected by the number of samples, compounds, or other reagents to be drawn and dispensed, the number of stations 121-124 in use on platform 129 for a particular application, the number and length of mixing operations to be conducted, the rapidity with which discrete sample mixtures are injected into the analysis apparatus, and other factors.

Arm 181 may address compound plate stations 122 used for agonist mode, antagonist mode, allosteric modulator mode, or various other operational or experimental modalities and protocols. Compounds or reagents may be taken up into pipette tip 188 and added to cell samples or buffer (for dilution purposes) in a predetermined or selected well of a microwell plate at a selected station 122. Mixing of cell sample material and compound or compound and buffer may be performed by arm 181 and probe 183, for example, through selective use of syringe 153 alternatively to draw a mixture from a microwell and to expel the mixture. In some embodiments, a single such cycle may be sufficient to provide adequate mixing, though a mixing cycle may be omitted in some instances, for example, or repeated for any desired number of iterations.

Specifically, arm 181 and probe 183 may address a suspension of viable cell samples and subsequently draw a selected or predetermined sample volume of evenly suspended cells into pipette tip 188 for delivery to a selected well of the microwell plate, i.e., arm 181 and probe 183 may be used to dispense the cell sample volume into microwell plate. Further, arm 181 and probe 183 may be implemented to mix the contents of a specific well (for example, by pipetting up and down a selected or predetermined number of times) without substantially disturbing the cells in the context of the parameters to be measured (e.g., intracellular Ca2+). Alternatively, the injection of cell samples into the well may be sufficient for mixing, eliminating the need for additional pipetting. The cell suspension mixture may then be left in the mixing well until the contents are withdrawn by arm 182 and probe 184 for injection to an analysis apparatus.

After mixing the cell samples and compound for a particular well (i.e., preparing a discrete sample mixture), arm 181 may then travel to waste bag station 124 and automatically eject pipette tip 188 from probe 183. In some embodiments, tip ejection may be monitored, for example, by an IR or other suitable sensor or camera to ensure proper and complete ejection of pipette tip 188. In the case of incomplete ejection, buffer may be rapidly flushed through probe 183 and pipette tip 188, and ejection procedures may be repeated until pipette tip 188 is removed from probe 183. Following confirmation of proper tip ejection, arm 181 may be manipulated to return probe 183 to tip rack 121 (or to a different tip rack) to retrieve a new pipette tip 188 in preparation for the next task.

As noted above, arm 182 and probe 184 may withdraw the cell material and compound (a discrete sample mixture) into a pipette tip 188 after an appropriate, predetermined, or otherwise selected duration following mixing; arm 182 and probe 184 may then engage pipette tip 188 with sample injection guide 139 (as illustrated in FIG. 5) and transfer the discrete sample mixture to flow cytometer 190 (or to another sample analysis apparatus).

Regarding injection of discrete sample mixtures into an independent fluidic system, it is noted that FIGS. 9, 10, 11, and 12 are simplified diagrams illustrating perspective, plan, side elevation, and axial cross-section views, respectively, of one embodiment of a sample injection guide. Additionally, as noted above, FIG. 5 is a simplified diagram illustrating a perspective view of one embodiment of a sample injection guide engaged with a pipette tip during use.

Guide 139 and its various components may be fabricated of virtually any suitably non-reactive material. In this context, “non-reactive” generally refers to materials which will not adversely affect the experimentation occurring in the analysis apparatus. In one embodiment, for example, guide 139 may be fabricated of DELRIN™ plastic, though other plastics, acrylics, fiberglass, metals, and other materials may also be suitable.

As indicated in the drawing figures, one embodiment of guide 139 may generally comprise a guide well 135 dimensioned and operative to receive or otherwise sealingly to engage pipette tip 188, and a port 136 in fluid communication with both guide well 135 and the fluidic system of the analysis apparatus. During injection operations, pipette tip 188 may be engaged or seated in guide well 135 such that liquid or air cannot leak through the area of contact between guide well 135 and pipette tip 188. In that regard, it will be appreciated that the general constitution and specific dimensions of guide well 135 (e.g., depth, internal diameter, and taper) may be selected in accordance with the type of pipette tip 188 with which it is intended to be used. For example, guide well 135 is illustrated as tapered in FIGS. 11 and 12; in some embodiments, taper or angular dimensions provided for guide well 135 may be specifically designed to cooperate with a corresponding and complementary tapered portion of pipette tip 188.

When pipette tip 188 is engaged with guide well 135 as set forth above, a discrete sample mixture, or other contents of pipette tip 188, may be injected through port 136 into the fluidic system of the analysis apparatus. Port 136 may be coupled to an independent fluidic system, for example, using flexible tubing, hose barbs, quick-disconnect assemblies, and other types of fluid coupling hardware and mechanisms generally known in the art. This “connection” between port 136 and the independent fluidic system has been omitted from the drawing figures for clarity.

When pipette tip 188 is withdrawn from guide well 135, the free stream dynamic pressure of the independent fluidic system may force liquid back through port 136 and into guide well 135, flushing the connection, port 136, and guide well 135. This flushing may prevent residual material from one discrete sample mixture from contaminating a subsequent discrete sample mixture and altering or otherwise affecting the analysis thereof. It will be appreciated that the dynamic pressure associated with the fluidic system may cause flooding and overflow of guide well 135; additionally, removing liquid back flushed through port 136 into guide well 135 may facilitate minimization of deleterious contamination between successive sample mixtures. Accordingly, some embodiments of guide 139 may additionally comprise an overflow well 134 and siphon ports 137,138.

During operation, back pressure from the independent fluidic system generally causes fluid to flush through port 136 and into guide well 135 and overflow well 134. The depth of fluid in guide well 135 and overflow well 134, on the other hand, may exert sufficient hydrostatic pressure to balance the pressure of the fluid entering wells 135,134 through port 136, preventing a spray or “geyser” effect and minimizing liquid waste. Back flushed liquids (and any sample cells, reagents, or other contamination carried therein) may be siphoned, either by gravity alone, for example, or by pumping mechanisms, through siphon ports 137,138.

It will be appreciated that the structural characteristics, relative dimensions, locations, and orientations of the various elements (i.e., wells 134,135, ports 136-138, and siphon pumps, if implemented) may be selected in accordance with the type of independent fluidic system employed and the operational dynamic pressures expected. For example, an additional siphon port may be required in some instances; alternatively, one or both of siphon ports 137,138 may be omitted. Where no siphon ports are provided, guide well 135 or overflow well 134 may simply be allowed to overflow into a waste drain or bag, for example, or a siphon tube which is not integrated into the structure of guide 139 may be employed.

In the exemplary embodiment, for instance, excess liquid not siphoned from overflow well 134 by siphon ports 137,138 may be directed to a channel 131, where it may then be drained to an appropriate waste container or drain through ports 132,133. Additionally or alternatively, one or both of ports 132,133 may be employed, for example, as guide holes for screws, bolts, or other fastening members, to facilitate attachment of guide 139 to platform 129 or to the analysis apparatus. The present disclosure is not intended to be limited by the structural configuration and design characteristics of guide 139 illustrated in FIGS. 5 and 9-12. It will be appreciated that numerous alterations may be made to guide 139, and that the functionality described herein not limited to the design depicted in the drawing figures.

In accordance with the exemplary embodiment, guide 139 may satisfy the functional requirements set forth below. As best illustrated in FIG. 5, guide 139 may serve as a docking port between a pipette tip 188 containing a discrete sample mixture and an input port (not shown) of flow cytometer 190 or any other sample analysis apparatus employed in conjunction with system 100. In the case of flow cytometer 190, for instance, such an input port may be embodied in or comprise a tube in fluid communication with a flow nozzle or cuvette. Guide 139 may have particular utility in cases where hydrodynamic focusing between the discrete sample mixture (injected by pipette tip 188 through guide 139) and sheath fluid in the fluidic system of the analysis apparatus occurs at the input port of the analysis apparatus or just downstream thereof.

In particular, guide 139 may allow the contents of pipette tip 188 to be directly injected through port 136 into flow cytometer 190 (or to any independent fluidic system) on a discrete sample-by-sample basis. Operation of guide 139 enables contents of pipette tip 188 (i.e., a discrete sample mixture) to be treated as, and to behave as, the ideal sample stream described in conventional flow cytometry applications, i.e., where individual sample tubes are manually placed at the sample input station.

Additionally, guide 139 may permit rapid flushing of the sample input tubing (e.g., the input port of the analysis apparatus) to remove adherent compounds and residual sample material from the previous sample mixture. It will be appreciated that the tubing connecting guide 139 (at port 136) to the flow nozzle (i.e., associated with the fluidic system of the analysis apparatus) ideally needs to be washed free of contamination between successive discrete samples; such flushing may prevent sample carryover artifacts in the data stream. To achieve this flushing between successive discrete sample input operations, as set forth in detail above, port 136 and guide well 135 may be in continuous fluid communication with the normal sheath fluid used in the fluidic systems of standard flow cytometers. When pipette tip 188 is disengaged from guide well 135, the sheath fluid of the independent fluidic system (that is normally under positive pressure) washes backwards through port 136. This reverse flow serves to wash the connector tube and the port 136. As set forth above, excess fluid may be removed by gravity, for example, or by continuous aspiration (such as by a vacuum pump) through siphon ports 137,138 and channel 131.

As set forth in detail above, guide 139 may facilitate docking or engagement of pipette tip 188 and guide well 135, allowing pipette tip 188 to be firmly and tightly sealed with the walls of guide well 135; additionally, guide 139 may be operative to prevent the force of docking (i.e., the engagement of pipette tip 188 with guide well 135) from disturbing the alignment between the cells in the sample mixture stream and the lasers of flow cytometer 190 or other equipment in the analysis apparatus. In some embodiments, the foregoing alignment may be achieved by utilizing a length of flexible tubing that communicates sample mixtures from port 136 to the independent fluidic system. Such flexible tubing may absorb stresses associated with repeated engagement of pipette tip 188 with guide well 135, and may prevent transmission of those stresses to components of the analysis apparatus. Maintaining alignment in the foregoing manner may ensure continuous data consistency and quality throughout repeated runs of successive experiments.

Delivery of a discrete sample mixture to the analysis apparatus may be controlled by the pipetting syringe 154 operatively coupled to probe 184 on arm 182 and, in turn, by a motor (such as a servo motor or equivalent device) driving syringe 154. Injection of a discrete sample mixture through port 136 may selectively be rapid and of brief duration, for example, or alternatively, slow and prolonged. In the exemplary embodiment, sample mixture injection rates may be selectively controlled, for example, through control of the servo motor, and thereby the dispense rate of syringe 154. Similarly, pipetting functionality for arm 181 and probe 183, including volumes and rates, may be controlled by a servo-motor driving syringe 153. As set forth above, such control may be effectuated through appropriate programming instructions for processing component 170.

When an injection cycle is completed (i.e., a discrete sample mixture has been injected through guide 139 to an independent fluidic system) arm 182 and probe 184 may move to a waste bag station 124 and eject pipette tip 188 to a waste container substantially as described above with reference to arm 181 and probe 183. As with the foregoing ejection procedure, ejection of pipette tip 188 from probe 184 may be monitored (e.g., by a sensor or camera) to ensure successful ejection of pipette tip 188. Respective arms 181,182 and probes 183,184 may be prepared for the next cycle by retrieving new pipette tips 188 from designated or selected tip racks 121.

In accordance with FIG. 15 embodiment, cell sample material to be analyzed may be maintained in suspension by an active cell suspension system (CSS) 140. During operation, CSS 140 may prevent the cells from settling and, accordingly, may keep cell material at a constant density throughout the entire suspension volume. In that regard, CSS 140 may generally comprise a tube 141 mounted to a rocking apparatus 145. Tube 141 may be loaded with cells and a liquid suspension medium, and generally comprises an aperture 142 allowing access to the contents thereof by pipette tip 188. Tube 141 and its contents may be rocked by rocking apparatus 145 from an horizontal position alternately to positions approximately +/−45 degrees off the horizontal axis. In some instances, rocking may be controlled such that CSS 140 does not agitate the suspension in such a manner as to perturb resting cell physiology as measured by fluorescent probes that indicate, for example, Ca2+i membrane potential or plasma membrane integrity.

By way of example, a suspension vessel, such as tube 141, may be a 50 ml sealable plastic tube (e.g., as may be available from Falcon Labware or various other manufacturers), though specific dimensions, volume, and material may be varied as desired. As noted above, tube 141 generally comprises an access port or aperture 142 allowing pipette tip 188 coupled to probe 183 to access the cell suspension in tube 141. In some embodiments, CSS 140 in general, and rocking apparatus 145 in particular, may be under control of processing component 170; responsive to an appropriate control signal from processing component 170, for example, operation of rocking apparatus 145 may be interrupted, and tube 141 may be maintained in a desired orientation, while pipette tip 188 coupled to probe 183 approaches tube 141, enters aperture 142, and withdraws a selected volume of cell sample material. Responsive to an additional signal from processing component 170, or following a predetermined or selected duration, rocking action may be resumed following withdrawal of pipette tip 188 from aperture 142.

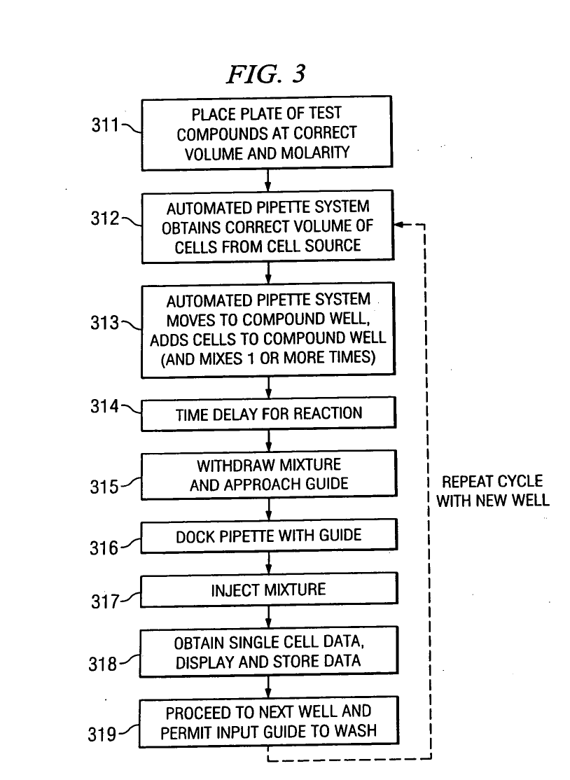

FIG. 3 is a simplified flow diagram illustrating the general operation of one embodiment of a method of performing an analysis using a direct sample injection system. At the initiation of any particular analysis method, as indicated at block 311, a plate of test compounds (at any desired or selected volume and molarity) may be placed at a selected or predetermined station 122 on platform 129; additionally or alternatively, a rack of test tubes, each of which may contain one or more compounds of a selected volume and molarity, may be placed at a selected or predetermined station 123 on platform 129. As set forth above, any number of microwell plates or test tube racks containing various compounds or reagents, or desired combinations thereof, may be placed at one or more such stations 122,123 on platform; specifically, the operation depicted at block 311 may be repeated as desired any number of times and in accordance with a particular analysis protocol. Locations (i.e., at stations 122 or 123 on platform 129) of specific microwell plates or test tubes, as well as the specific contents of each well or test tube and associated data and parameters, may be input or otherwise recorded, for example, using software or other instruction sets, in processing component 170 for further reference, to program sequences of operations executed by arms 181,182 and probes 183,184, and the like.

As indicated at block 312, an automated pipetting apparatus (such as liquid handler 180, for example) may obtain a predetermined or preselected volume of cell material and suspension medium (e.g., from CSS 140). In some embodiments, instructions governing or otherwise influencing the operation depicted at block 312 may be provided by processing component 170 or an equivalent controlling mechanism adapted to provide commands to automated or semi-automated electromechanical systems; additionally or alternatively, such instructions may be provided, in whole or in part, in accordance with user intervention. In the exemplary FIG. 14 implementation, such retrieval of sample cell material may be effectuated by a dedicated pipetting arm 181 and associated hardware, though various other pipetting arm implementations are also contemplated.

Notwithstanding which of a plurality of pipetting arms (such as arms 181,182, for instance) performs the operation at block 312 (or whether a single arm liquid handler 180 is employed), sample material may be added or provided to a specified or predetermined compound well (at station 122) or test tube (at station 123) as indicated at block 313. Specifically, the operation at block 313 represents preparation of a discrete sample mixture (i.e., a mixture comprising a desired volume of sample material obtained from a common sample source (such as from suspension vessel or tube 141, for example) and a specified or preselected compound, reagent, buffer solution, or some desired combination thereof) at a specified location (e.g., at station 122 or station 123) on platform 129. As further indicated at block 313, one or more mixing operations may be conducted. In some instances (depending, for example, upon analysis protocols, the specific chemistry of discrete sample mixtures, and other factors), the foregoing providing sample material to a well or test tube may also effectuate necessary or desired mixing. Alternatively, mixing may be performed through one or more pipetting cycles wherein the discrete sample mixture (of sample material and compound or other chemical components in selected well or test tube) is alternately withdrawn and subsequently returned to the appropriate well or test tube. Again, the operation depicted at block 313 may be influenced or controlled by processing component 170, either automatically or in accordance with user intervention, and driven by a pump system (such as represented by reference numeral 151 in FIG. 15).

As indicated at block 314, a time delay may be provided to allow sufficient time for desired reactions to take place for a particular discrete sample mixture. In some embodiments, such a delay time may be identical, or substantially so, for each discrete sample mixture prepared as set forth above. Alternatively, reaction time durations for one or more discrete sample mixtures may vary from other discrete sample mixtures prepared on platform 129 and awaiting injection into the analysis apparatus. It will be appreciated that synchronization considerations, prioritization strategies, or both, for pipetting arm motions may be influenced or otherwise affected in accordance with the various reaction times required by, or desired for, each discrete sample mixture to be prepared and provided to the analysis apparatus. Accordingly, delay times may be recorded and monitored by processing component 170, for example, and liquid handler 180 may be controlled appropriately to accommodate various reactions and delay durations.

Following a desired or predetermined delay period (block 313) a discrete sample mixture may be withdrawn from its well or test tube station (122 or 123) for delivery or approach to sample injection guide 139 as indicated at block 315. Specifically, each discrete sample mixture prepared in a particular location on platform 129 may be individually addressed and withdrawn successively by liquid handler 180 in accordance with instructions provided, for example, by processing component 170. As illustrated in the drawing figures and described in detail above, an exemplary direct injection system may employ a clean pipette tip 188 for the operation depicted at block 315, eliminating or minimizing contamination between successive injection operations (blocks 316 and 317).

As indicated at blocks 316 and 317, a discrete sample mixture may be injected into the fluidic system of an analysis apparatus substantially as set forth above with specific reference to FIGS. 5 and 9-12. In particular, a pipette tip 188 containing a discrete sample mixture may be docked or sealingly engaged with a sample injection guide 139 (block 316); the discrete sample mixture may then be provided through guide 139 to an independent fluidic system (block 317) associated with a sample analysis apparatus (such as flow cytometer 190). As noted above, an injection rate for a particular discrete sample mixture may be selectively controlled, for example, through operation of a pump system (such as indicated at reference numeral 152) under control of processing component 170.

Data regarding a discrete sample mixture may be recorded, for example, on computer readable media at processing component 170, at another electronic device, or both, for storage or analysis; additionally, such data may be transmitted, via recording media or network data transmissions, for instance, to any desired computerized device or data processing apparatus for recordation or for further analysis. Appropriate, desired, or relevant data relating to the foregoing operations described with reference to blocks 311-315 and 317 may include, but not be limited to, some or all of the following information associated with a particular discrete sample mixture: specific chemistries, volumes, percentages, concentrations, compositions, or other factors related to the discrete mixture of cell samples, compounds, reagents, and buffer solutions; mixing parameters such as the number of pipetting cycles performed, for example, and the forcefulness or rapidity (in terms of fluid flow rates, for example) with which those cycles were executed; the time delay allowed between preparation of the discrete sample mixture and injection of same to the analysis apparatus; the time at which the particular discrete sample mixture is injected into the analysis apparatus, as well as the rate (or duration) of the injection process; and any other parameter monitored or controlled by processing component 170. It will be appreciated that the nature and relevance of data recorded in conjunction with the foregoing processes may be a function of the particular experiment or assay occurring in the analysis apparatus.

Further data may be obtained in accordance with standard or modified operation of the analysis apparatus as indicated at block 318. Though the present disclosure is not intended to be limited to any particular analysis apparatus, or to the operational characteristics or limitations thereof, it is noted that the operation depicted at block 318 may be executed by a flow cytometer 190, for example, or by any other sample analysis equipment known in the art or developed and operative in accordance with known principles of fluidic systems. Data acquired by the analysis apparatus (block 318) may be combined or otherwise associated with the data recorded as set forth above (in conjunction with blocks 311-315 and 317) at processing component 170 or elsewhere; alternatively, separate data files may be maintained for storage or processing as desired.

As indicated at block 319 and the dashed line returning to block 312, the foregoing operations may be executed any number of times, and for any number of discrete sample mixtures sought to be analyzed. As set forth above, processing component 170, or equivalent mechanisms, may be used to record the locations of discrete sample mixtures prepared, and those which have been analyzed versus those that have not.

As set forth above, guide 139 and any attendant coupling tubing or other fluid conduit connecting same to the independent fluidic system may be washed, for example, through a back flush of sheath fluid through operative portions of guide 139. This wash operation, set forth above with specific reference to FIGS. 5 and 9-12, is also depicted at block 319.

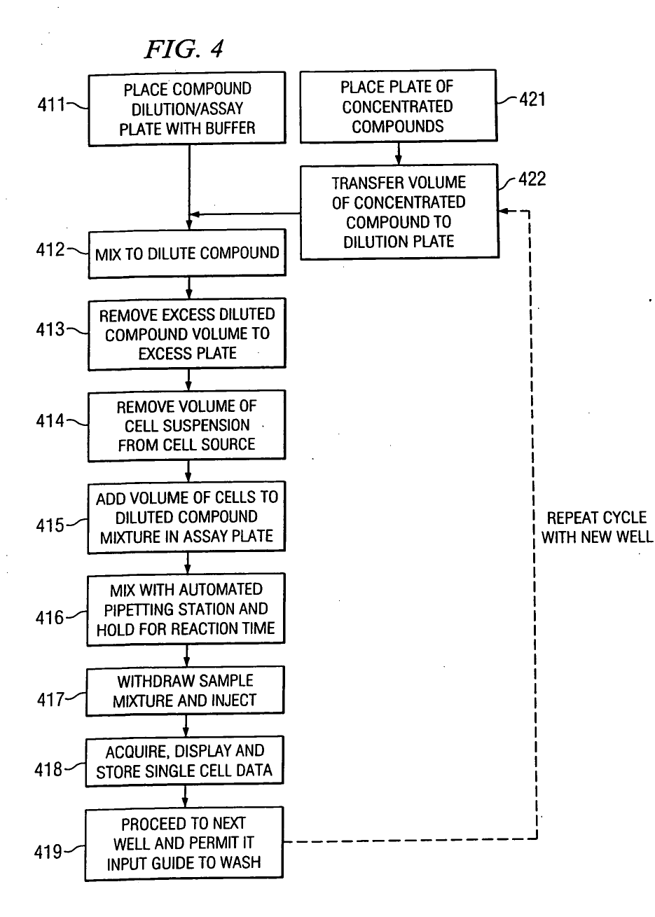

FIG. 4 is a simplified flow diagram illustrating the general operation of another embodiment of a method of performing an analysis using a direct sample injection system. At the initiation of any particular analysis method, as indicated at blocks 411 and 421, various plates or racks of test tubes containing compounds and buffer solutions (at any desired or selected volume and molarity) may be placed at selected or predetermined stations 122,123 on platform 129. As with the method described above, any number of microwell plates or test tubes containing various compounds, reagents, buffers, or desired combinations thereof, may be placed at one or more such stations 122,123 on platform. Appropriate data representative of locations of specific microwell plates or test tubes, as well as the specific contents thereof, may be input or otherwise recorded at processing component 170 or elsewhere. These data may be employed for further reference, to program sequences of operations executed by arms 181,182 and probes 183,184, and the like.

As indicated at blocks 412 and 422, an automated pipetting apparatus (such as liquid handler 180, for example) may transfer one or more compounds to selected other wells or test tubes at specified locations on platform; the resulting combination of liquids may be mixed as indication at block 412. In some embodiments, instructions governing or otherwise influencing the operations depicted at blocks 412 and 422 may be provided by processing component 170 or an equivalent controlling mechanism; additionally or alternatively, such instructions may be provided, in whole or in part, in accordance with user intervention. Mixing at block 412 may proceed substantially as set forth above with specific reference to block 313 in FIG. 3.

Following mixing of desired components, excess liquid may be removed from a specific well or test tube (block 413) to ensure that the particular well contains an appropriate amount of compound, reagent, buffer, and the like, for creating the desired discrete sample mixture for that particular well or test tube. Excess liquid withdrawn as contemplated at block 413 may be discarded as waste. The operation depicted at block 413 may be selectively controlled in accordance with desired sample analysis protocols for a particular experiment, in whole or in part, by processing component 170.

The operations depicted at blocks 414-416 (i.e., removing or obtaining a desired volume of cell sample material from a source such as CSS 140, for example, adding same to a desired well or test tube, mixing, and allocating a desired delay time), may proceed substantially as set forth above with specific reference to blocks 312-314 in FIG. 3. Specifically, the operations at blocks 414-416 represent preparation of a discrete sample mixture comprising a desired volume of sample material obtained from a common sample source (such as from suspension vessel or tube 141, for example) and a specified or preselected compound, reagent, buffer solution, or some desired combination thereof. This discrete sample mixture may be prepared and maintained at a specified location (e.g., at station 122 or station 123) on platform 129.

As further indicated at block 416, one or more mixing operations may be conducted. Such operations may depend, for example, upon analysis protocols, the specific chemistry of discrete sample mixtures, and other factors substantially as described above. Mixing may not be required in some applications. Further, a time delay may be provided to allow sufficient time for desired reactions to take place for a particular discrete sample mixture. While such a delay time may be identical, or substantially so, for each discrete sample mixture, reaction time delays for one or more discrete sample mixtures may vary from other discrete sample mixtures. Accordingly, synchronization considerations, prioritization strategies, or both, for pipetting arm motions may be influenced or otherwise affected. Where required, one or both of the operations depicted at block 416 may be influenced or controlled by processing component 170, either automatically or in accordance with user intervention.

The operations depicted at blocks 417-419 (i.e., withdrawing and injecting a discrete sample mixture, acquiring data from an analysis apparatus, and reiterating the procedure), may proceed substantially as set forth above with specific reference to blocks 315-319 in FIG. 3. In particular, a discrete sample mixture may be retrieved by liquid handler 180 and injected (block 417) into the fluidic system of an analysis apparatus as described above with specific reference to FIGS. 5 and 9-12. In that regard, a pipette tip 188 containing a discrete sample mixture may be docked or sealingly engaged with a sample injection guide 139; the discrete sample mixture may then be provided through guide 139 to an independent fluidic system associated with a sample analysis apparatus (such as flow cytometer 190). An injection rate or duration for a particular discrete sample mixture may be selectively controlled, for example, through operation of a pump system (such as indicated at reference numeral 152) under control of processing component 170.

Relevant or desired data associated with a discrete sample mixture may be recorded, transmitted, or both, for example, under control of processing component 170 substantially as set forth above. As in the FIG. 3 embodiment, these data may include: specific chemistries, volumes, percentages, concentrations, compositions, or other factors related to the discrete mixture of cell samples, compounds, reagents, and buffer solutions; mixing parameters; the time delay; the time (and rate) at which the particular discrete sample mixture is injected into the analysis apparatus; and any other parameter monitored or controlled by processing component 170. The nature and relevance of data acquired, recorded, or otherwise manipulated in conjunction with the foregoing processes may be a function of the particular experiment or assay occurring in the analysis apparatus.

Additional data may be acquired in accordance with standard or modified operation of the analysis apparatus as indicated at block 418. Finally, as indicated at block 419 and the dashed line returning to block 422, the foregoing operations may be iterated any number of times, and for any number of discrete sample mixtures sought to be analyzed. Processing component 170, or equivalent mechanisms, may be used to record the locations of discrete sample mixtures prepared, and those which have been analyzed versus those that have not. Guide 139 and any attendant coupling or fluid conduit connecting same to the independent fluidic system may be washed, for example, through a back flush of sheath fluid through operative portions of guide 139. This wash operation, set forth above with specific reference to FIGS. 5 and 9-12, is also depicted at block 419.

The specific arrangement and organization of functional blocks depicted in FIGS. 3 and 4 are not intended to be construed as implying any particular order or sequence of operations to the exclusion of other possibilities. Alternative sequences, combinations and simultaneous execution of various operations are also contemplated, and may be enabled or facilitated, for example, in multiple arm liquid handler embodiments and during successive iterations of sample injection cycles. For example, the operations depicted at blocks 315-319 with respect to one sample mixture may occur in parallel, or substantially simultaneously, with operations 312-314 conducted with respect to a different or subsequent iteration for a next successive or different discrete sample mixture. Similarly, the operations depicted at blocks 422 and 412-416 (with respect to one sample mixture) may be executed in parallel, or substantially simultaneously, with the operations depicted at blocks 417-419 (with respect to a sample mixture previously prepared). Those of skill in the art will appreciate that the operations depicted at blocks 317 and 318 may occur substantially simultaneously; similarly, the injection operation (block 417) and the acquisition operation (block 418) depicted in FIG. 4 may also be executed substantially simultaneously.

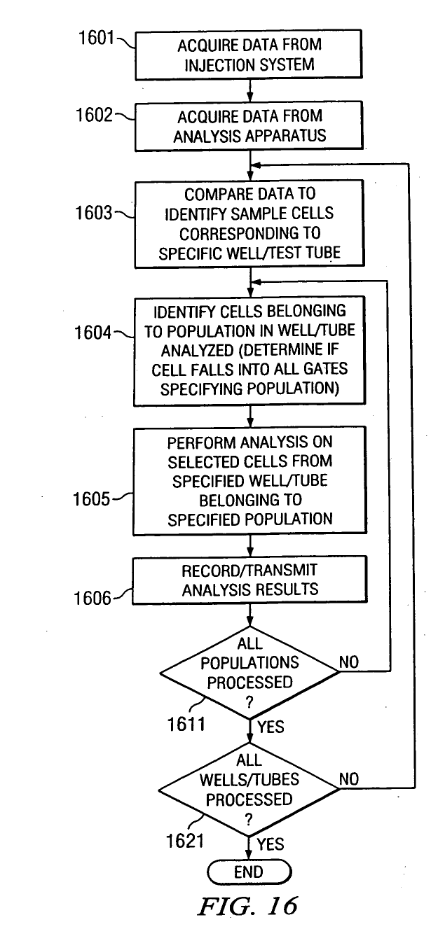

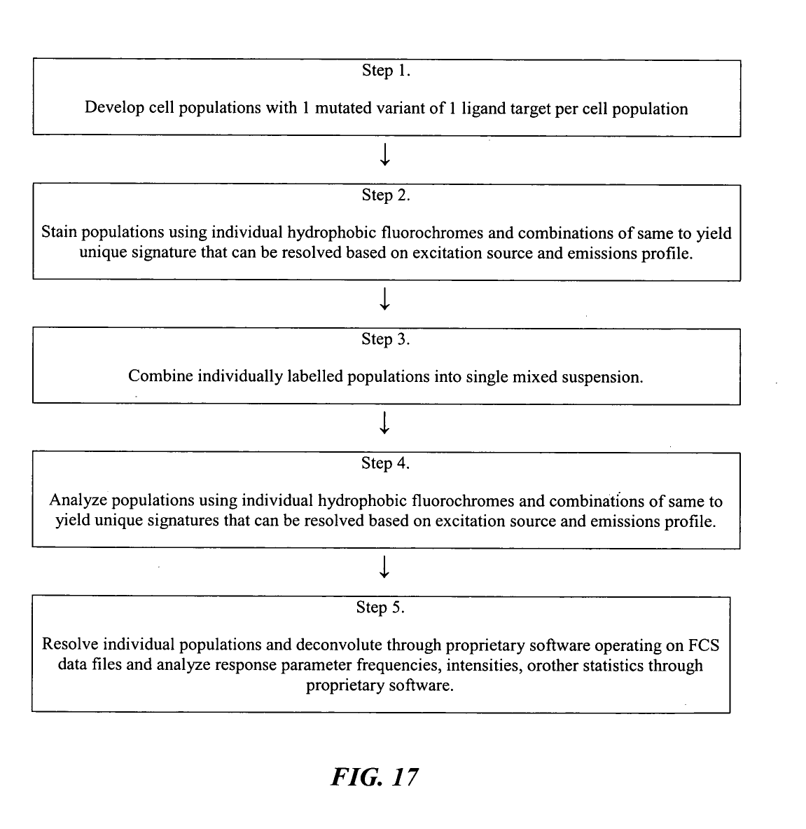

FIG. 16 is a simplified flow diagram illustrating the general operation of one embodiment of a method of performing an analysis. As indicated at blocks 1601 and 1602, data may be acquired from a sample injection system (such as by processing component 170, for example) and from an analysis apparatus substantially as set forth above with specific reference to FIGS. 3 and 4. Acquired data may then be compared (block 1603) to identify which data records obtained by the sample analysis apparatus correspond with data records obtained and recorded by the injection system associated with a particular discrete sample mixture. Where an injection time and rate for a particular sample mixture are recorded by processing component 170, for example, data acquired by the analysis apparatus at that time and for a specific duration thereafter may be flagged as associated with that particular discrete sample mixture. In the foregoing manner, data from the analysis apparatus may be correlated with data from the injection system such that data records may be matched and associated with a specific discrete sample mixture. This correlation may be have particular utility in ascertaining which analysis results are obtained from the sample mixture in a particular well or test tube; in some applications, correlating analysis results with the composition of a sample mixture may facilitate interpretation of the results.

As indicated at block 1604, cell sample material belonging to a particular population may be identified and associated with a specific well or test tube from which the sample mixture was prepared and drawn. In accordance with one embodiment, for example, the identification of cells within a population may comprise determining if a cell falls into all gates specifying the population sought to be identified. It will be appreciated that these gates, and other sorting criteria or parameters, may be user-specified and application specific. In the foregoing manner, cells within a particular well or test tube may be associated with the population criteria appropriate or desired for a particular experiment.

A selected or desired analysis may then be performed on selected cells from a particular well or test tube (i.e., discrete sample mixture) that are identified as belonging to or associated with a particular population as indicated at block 1605. Various analyses including statistical analytical techniques are contemplated at block 1605. For example, mean intensity, median intensity, percentage of cells exceeding a predetermined threshold intensity value, and the like, may be appropriate or desired. It will be appreciated that the nature of the analysis performed at block 1605, as well as the nature of the data records acquired in conjunction with its execution, may vary in accordance with some or all of the following, without limitation: the type of analysis apparatus employed; the functional characteristics and limitations thereof; the operational modality or parameters set to control the analysis apparatus; the type of experiment conducted; and other factors.

Data acquired during the analysis at block 1605 may be recorded, transmitted, processed, or otherwise manipulated as generally indicated at block 1606. Recorded data records may be saved or stored, for example, on computer readable media for processing at a later time; additionally or alternatively, data processing may occur simultaneously or in conjunction with the recordation depicted at block 1606. As set forth above with reference to FIGS. 3 and 4, data may be transmitted via recording media, for instances, or via network data communications to any desired computerized device or processing apparatus.

As indicated by the decision blocks 1611 and 1621, the foregoing process may be selectively iterated, for example, until all populations and all discrete sample mixtures have been analyzed. The iterative nature of the FIG. 16 embodiment may be selectively interrupted in accordance with user intervention if desired.

The entire contents of all references, patents, and patent applications cited in the present disclosure are hereby explicitly incorporated by reference in their entirety.

### EXAMPLES

### Example 1

### Developing and Staining Cell Populations with 1 Mutated Version of 1 Ligand Target Per Cell Population One at a Time

Step 1. Developing variant ligand targets. Variant, or mutant, ligand targets were developed using molecular biology techniques to mutate the target cDNA at one or more residues. This was performed in a shotgun or combinatorial fashion or specifically in a site-directed mutagenesis fashion.

Step 2. Development of color-coded populations for flow cytometry (FCM) analysis and sorting. Regardless of the transfection methodology used, each transfected population was individually color-coded and was discretely recognized by the instrument during analysis. The color-coding of each population was achieved by staining each population separately. Each population was labelled by one or both of two different methods. The first method used hydrophobic fluorochromes such as DiI, DiO, DiA, DiD and DiR (Invitrogen, Carlsbad, Calif.) that distribute into the lipid membranes of the cells, and other non-toxic fluorochromes that partition into various cell compartments. Each population was supended at approximately 1×10⁶cells/ml in a supportive medium such as their normal growth medium or Hybridoma Medium (Sigma-Aldrich). The fluorochromes were added from a stock solution (e.g., 10 mM) to a final concentration of about 0.1-10 uM. Cells were maintained in suspension for 60-90 minutes on a rotator device and wrapped in foil to block ambient light. Afterwards the cells were pelleted by centrifugation, washed once in the appropriate media and resuspended for use in the assay. The second method uses the indirect, two-step staining technique. The cells are suspended at 0.1×106 to 10×106 cells per milliliter in a supportive medium (e.g., Hybridoma medium) and incubated with X-biotinylated phosphoethanolamine (biotin-X-DHPE, Invitrogen, Carlsbad Calif.) from a stock solution of 1-10 mg/ml, to a final concentration of 0.5 to 10 μg/ml. The cells are wrapped in foil and placed on a rotator platform at room temperature for 60 minutes. The cells are pelleted and washed in the media, and incubated with a second molecule such as streptavidin-conjugated Alexa 488 (Invitrogen) at 1-20 ug/ml for 60 minutes on the rotator. Cells are then pelleted and washed once before analysis. The concentration of stain, the cell density and the duration of the staining step were separately optimized for different cell types and instruments to yield the clearest resolution between each fluorescent population during data analysis. Typically the best inter-population resolution was achieved with the brightest and most homogeneous staining.

Step 3. Combining cell populations After staining, the cells from each cell population were combined to provide a mixed cell suspension, wherein each cell showed the distinct color-coded fluorescent signature of its source cell population when single cells were excited by different laser sources and the emissions were collected through different optical filters.

Step 4. Analyzing populations by flow cytometry. The combined cell populations were mixed with a ligand by an automated cell-compound mixing system and analyzed by an automated sample input flow cytometry system (FCM). Cells and compound were mixed thoroughly so that all cells were exposed to ligand within 2 seconds or less (mix delay). The mixture was then injected to the FCM within 5 seconds (injection delay), or more if desired. The cells were injected over a period of 5 seconds (injection period), or longer if desired. The analysis was performed so that one or more laser beams in the FCM, depending on the instrumentation used, excite each cell in the mixture. Fluorescence emissions were collected down the emissions collection pathway of the instrument and a specific spectral region of a particular fluorochrome's emissions was selected for transmittance to the detector using spectral steering mirrors, dichroics and filters. It was not necessary that each fluorochrome be excited optimally nor that the emission(s) for each fluorochrome be collected at the optimum emissions wavelength. The goal was to combine as many fluorochromes as possible in the mixed population and resolve them from each other based on the final collected emissions profile. Thus, it was possible to combine the results from several fluorochromes with similar, but not identical, emissions profiles into a single dual emissions (bivariate) plot.

Step 5. Resolving populations and deconvoluting data. After the data were collected in FCS standard file format, the populations were thoroughly resolved from one another using logical gating analysis software. For example, as shown in FIG. 19, the Alexa 594 population was isolated in software by building regions around each of the other populations as identified in the other three bivariate dot-plots in the figure (see FIG. 19.C.) and removing those populations from the display, leaving only the dot-plot corresponding to the Alexa 594 population apparent in the bivariate display (see FIG. 19.D.). Isolating the results from the Alexa 594 population made it easier to analyze the response of that population. More detail is provided in Example 3, below. Once the populations were clearly resolved, analysis of the responses (e.g., Ca²⁺ᵢ mobilization) was performed for each population separately and the results were recorded to a file.

### Example 2

### Resolving Twenty (20) Populations of U937 Cells

U237 cells, which express an ATP-activated purinergic receptor, were used to develop 20 cell populations. The 20 discrete U937 cell populations were directly stained with individual fluorochromes and combinations of fluorochromes, using DiO, DiI, DiA, DiD, DiR, FM 1-43 and FM 4-64. The fluorochromes were solubilized in dimethyl sulphoxide (DMSO), dimethylformamide (DMF) or ethanol at 1-10 mM to prepare a stock solution. The cells were suspended in serum-free Hybridoma Medium (Cat. No. H 4281, Sigma Aldrich) as a staining buffer. Each cell population was stained separately by diluting the cells to approximately 10⁶ cells/ml and adding the dyes to final concentrations of 1-10 μM. All cells were also loaded with Indo-1 to monitor Ca²⁺ mobilization in all populations in response to stimulation of cells with ATP. The indicator dye was loaded into the cells by adding the acetoxy methyl ester (AM) form of the dye at 0.5 to 10 μM, from a stock solution of 1-5 mM in DMSO, to the cell suspension while the cells were being stained with color-coding dyes. The total DMSO concentration was kept at 1% or less, by volume, in the cell suspension. Cells were wrapped in foil to prevent photobleaching of the probes by ambient light, and were placed on a rotating or rocking platform to keep the cells in suspension. The cells were incubated 30 to 90 minutes (60 minutes is typical) in suspension at room temperature.

FIG. 18 illustrates the resolution of the twenty (20) color-coded U937 cell populations and the Ca²⁺ mobilization response in each population, as monitored by 3-laser flow cytometry. Cells were pooled and analyzed on a Cytomaton CyAn high-speed flow cytometer. Ca²⁺ mobilization in all of the populations was monitored with Indo-1 following stimulation of the cells with 10 μM ATP. Twenty (20) discrete populations were developed using DiO, DiI, DiA, DiD, DiR, FM 1-43 and FM 4-64. The emission profiles for DiI, DiO, DiA, FM 1-43 and FM4-64 were collected after excitation by a 488 nm argon laser source, and the emission profiles for DiD and DiR were collected after excitation by a 635 nm diode laser. The emissions were selected and steered into different bandpass filters using 550 nm and 600 nm dichroic mirrors for the 488 nm excitation path, and collected using the bandpass filters shown on the axes of each figure. For example, “530/40” as shown on the x-axis of FIGS. 18.D through 18.I. is a 530±40 nm bandwidth filter. For the 635 nm excitation path, the emissions were split using a 720 nm longpass steering dichroic mirror to direct the emissions to a 670 nm bandpass filter and 740 longpass filter, to collect all emissions above 740 nm.

### Example 3

### Resolution of Eight HEK293 Cell Populations Using the Two-Step “Anchor Molecule” Staining Technique

Eight (8) distinct color-coded populations were resolved simultaneously using the biotinylated-phosphoethanolamine technique and single cell flow cytometry (FCM). Eight different groups of HEK293 cells were first stained with biotinylated phosphoethanolamine (DHPE, Invitrogen, Carlsbad Calif.), then with streptavidin- or avidin-conjugated forms of Alexa™ fluorochromes (Molecular Probes, Inc., Eugene Oreg./Invitrogen, Carlsbad Calif.). Alexa 488, Alexa 500, Alexa 514, phycoerythrin, Alexa 594, Alexa 647, Alexa 660 and Alexa 750 or phycoerythrin (PE). Each HEK293 cell population was suspended in Hybridoma Medium at 1×106/ml, and 5 ug/ml of biotinylated DHPE was added from a stock of 10 mg/ml in DMSO. The cells were placed on a rotater platform for 60 minutes, and then pelleted by centrifugation and resuspended in Hybridoma Medium at 1×10⁶/ml. A different avidin- or streptavidin-conjugated fluorochrome was added to each sample, and they were maintained on the rotater for an additional 60 minutes. The cells were pelleted, washed in Hybridoma Medium, resuspended at 1×10⁶/ml in Hybridoma Medium and combined for analysis by FCM. The cells were also loaded with the Ca²⁺ᵢ indicator dye Indo-1 during the first incubation, and this indicator was excited by the multiline UV laser source. Each cell was also excited by a 488 m laser and a 647 nm laser. The emissions from the 488 and 647 nm lasers are shown here. Emissions derived from the 488 nm excitation were collected using optical steering mirrors (525 nm, 555 nm, and 605 nm dichroic mirrors) and finally filtered with 505+/1 10 nm, 540±20 nm, 580±30 nm and 613±nm bandpass filters as shown in FIG. 19. Emissions derived from the 647 nm excitation laser were steered with a 695 nm dichroic mirror and were collected through 670±20 nm and 787±nm filters. The populations were resolved using gating logic to identify and clarify specific populations from other contaminating fluorescent signatures. The Alexa 488, Alexa 500, Alexa 514 and PE populations were resolved using the 488 nm laser (see FIGS. 19.A. and 19.B). The Alexa 594 population, excited by the 488 nm laser, was partially obscured by the other populations as shown in panel 3.C. (613 nm by 505 nm). The Alexa 594 population could be resolved in software by creating regions on the bivariate plots shown in FIG. 19.A. (540 nm by 505 nm emissions) and FIG. 19.B. (580 nm by 540 nm emissions), and then excluding other populations through a 613 nm by 505 nm display (FIG. 19.D.). The Alexa 647, Alexa 660 and Alexa 750 populations were resolved using the 647 nm laser and a display of 787 nm by 670 nm emissions (FIG. 19.E.). Ca²⁺ᵢ mobilization responses to agonist ligands to endogenous receptors were not altered by any of the fluorochromes.

### Example 4

### Resolution of Ten Populations Using Two Fluorochromes, a Single Anchor Molecule and a Single Laser

A population of HEK 293 cells was divided into 10 individual populations. The ten populations were stained with 5 ug/ml Biotin-X DHPE at 1×10⁶ cells/ml in Hybridoma Medium for one hour on a rocking platform at room temperature. The Biotin-X DHPE-stained cells were pelleted, resuspended in Hybridoma Medium and then washed once and resuspended in Hybridoma Medium. One population (population #10) was not stained further. Streptavidin-conjugated Alexa 700 and Streptavidin-conjugated Alexa 635 were added to the remaining nine individual populations in the following ratios of Alexa 700:Alexa 635 to achieve differential staining of the populations with the two fluorochromes: Population #1, 0:10; Population #2, 1:9; Population #3, 3:7; Population #4, 1:1; Population #5, 7.5:2.5; Population #6, 9:1; Population #7, 25:1; Population #8, 75:1; Population #9, 10:0; Population #10, unstained. The final concentration of the Alexa stain preparations was 20 μg/ml. The populations were pelleted, reuspended, pelleted and resuspended in Hybridoma Medium to a final density of about 1×10⁶/ml. The 10 populations were mixed in equal proportions and analyzed by FCM using excitation from a 635 nm diode laser with 35 mW of power. Fluorescence emissions were collected with a 700 nm long pass steering dichroic, to collect the Alexa 700 emissions passing through the dichroic, and reflected light was filtered through a 665±20 nm bandpass filter to collect the A635 emissions.

### Example 5

### Multiplexed Analysis of Apoptosis and Necrosis in Four Different Cell Types Simultaneously

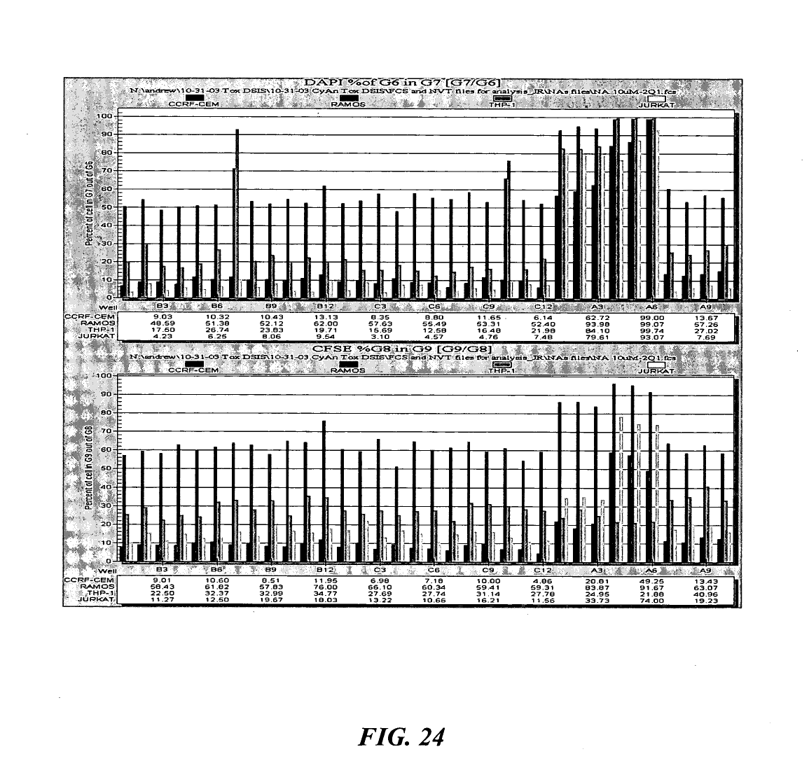

This example demonstrates how the system provided herein can be used to carry out multiplexed measurements of populations and response parameters simultaneously, resulting in a determination of the specificity of different compounds towards different cells (the affinity of the compounds) and the effect of the compounds on different cellular properties (the efficacy of the compounds). Four hematopoietic cell lines (CCRF-CEM, Jurkat, RAMOS, THP-1), all from ATCC, were stained with the fluorochromes DiD and DiR as described in Example 2 to color-code the four distinct cell populations. Cells were also loaded with the cytosolic probe CFDA SE (carboxyfluorescein diacetate, succinimidyl ester (5(6)-CFDAse, Invitrogen, Carlsbad Calif.) by incubating cells in 1-10 uM CFDA-SE in Hybridoma Media for 60 minutes at room temperature. CFDA SE allows tracking of cell division (generational analysis), as it is retained within the cells for days and is distributed evenly amongst daughter cells. Color-coded cells from each of the four populations were pooled in a mixed cell suspension and seeded at 0.5×10⁶ cells/ml into 96-well microwell plates. Each well contained test compound (ligand) at 10 uM, and wells 5 through 10 from the right contained positive buffer controls, while wells one through four from the right contained negative buffer controls. The plates were incubated overnight to allow the compounds to affect cellular proliferation and viability as measured by apoptosis. The following day, supernatants were removed and the cell nuclei were stained with 10 ug/ml of the membrane impermeant DNA probe DAPI (Invitrogen). DAPI staining can be used to distinguish cells with completely permeant plasma membranes, as seen in necrotic cells, from those with slightly leaky membranes, as seen in apoptotic cells. The plates were placed on the autosampling flow cytometry system as described above, and the contents of each well were analyzed to resolve the percentage of cells exhibiting evidence of apoptosis and necrosis (DAPI fluorescence) and the proliferative activity (CFDA SE fluorescence) in each of the four populations. As shown in FIG. 24, measurements of apoptosis and necrosis (DAPI fluorescence) in each of the four populations are shown in the top panel, and the proliferative activity (CFDA SE fluorescence) in each of the four populations is shown in the bottom panel. Each cluster of bars in the bar plots of FIG. 24 displays the results in the following order: left to right, CCRF-CEM, RAMOS, THP-1, Jurkat. The positive and negative controls are shown in the far right 10-bar clusters. The seventh compound from the left (top panel) promoted apoptosis/necrosis in the RAMOS and particularly the THP-1 cells with little effect on their proliferation (bottom panel), while the twelfth compound from the left (bottom panel) retarded the proliferation of the RAMOS cells with minimal induction of apoptosis/necrosis. This illustrates the ability of the system to simultaneously resolve selectivity of compound activity against specific parameters and against specific cells.

### Example 6

### Multiplexed Simultaneous Measurement of Ca²⁺ᵢ Mobilization Responses in Immunophenotyped Subsets of Primary Human Blood Cells

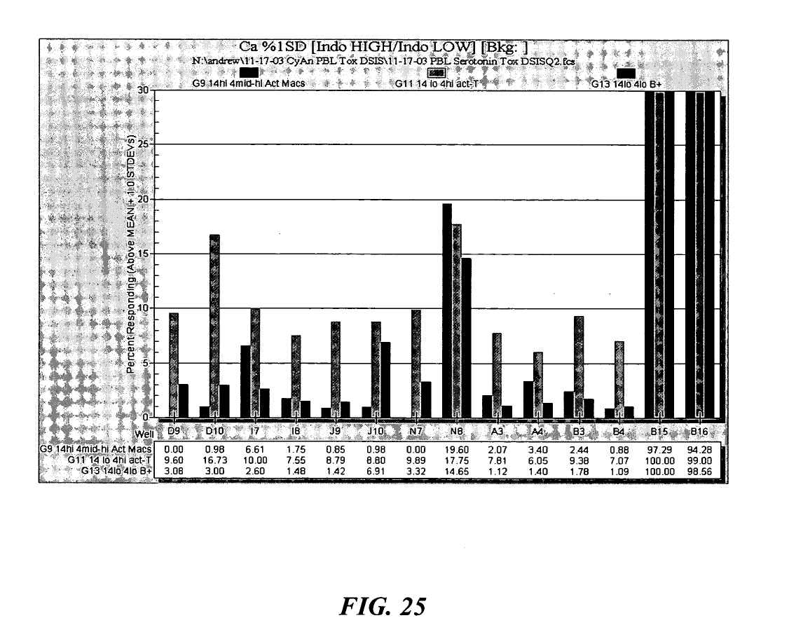

This example demonstrates how cellular subsets within primary tissue sample populations can be identified with antigen-specific monoclonal antibodies and analyzed for a rapid response parameter, to test compounds in an automated, multiplexed format. Here, the rapid response parameter is Ca²⁺ᵢ mobilization. Human blood was obtained and peripheral blood mononuclear cells (HPBMC) were isolated using density gradient centrifugation. The HPBMC were loaded with the Ca²⁺ᵢ indicator dye Indo-1 as described in Example 2, and then stained with Alexa 647-anti-CD4 and Alexa 700-anti-CD14 antibodies (Becton-Dickinson) to identify the CD4+ helper T cells and the CD14+ monocytes, respectively. Cells that were not stained by either fluorochrome-coupled antibody (double negative cells) were presumed to be CD14−/CD4− cells that did not belong to either population. By using antigen-specific flurochromes, it was possible to develop multiple color-coded cell populations, each having a distinct optical signature, without having to fractionate the heterogenous starting stample. Not only did this target-specific approach to color-coding cell populations make it possible to generate multiple distinct cell populations in a single heterogeneous starting sample, the approach automatically produced a mixed cell suspension suitable for multiplexed multitarget analysis. Finally, as described below and shown in FIG. 25, this approach made it possible to gain useful information about cells that were not stained by either antibody-coupled fluorochrome (the double negative cells).

The cells in the mixed cell suspension were then analyzed in an automated flow cytometry system that mixed the cells with test compounds taken from a 96-well compound storage plate, after which the mixtures were directly injected into the flow cytometer. In the flow cytometer, the Ca²⁺ᵢ mobilization response of each cell was measured and resolved, so that the Ca²⁺ᵢ mobilization response in individual subsets of cells (CD4+ helper T cells, CD14+ monocytes, and double negative cells) was determined in an automated manner.

The Ca²⁺ᵢ response was quantified by setting a threshold basal Ca²⁺ᵢ level and determining the percentage of cells that crossed the threshold during the analysis. As shown in FIG. 25, each cluster of bars along the x-axis indicates the response of the three populations to the test compound listed in the legend beneath the cluster. Each compound (ligand or control) is described using its position in the 96-well plate, and only a subset of the results was selected for purposes of illustration. In FIG. 25, each cluster of bars shows, from left to right, the percentage of CD14+ cells (medium shading), CD4+ cells (lightest shading), and double negative cells (darkest shading) that responded to the test compound. Cells were treated with Ca²⁺ᵢ ionophore (ionomycin) as a positive control: results for the ionomycin positive controls shown at B15 and B16, at the far right side of the histogram chart in FIG. 26. Cells were treated with buffer as a negative control: results for negative controls are shown at A3 and A4. Note that the negative control assays provide cell-specific baseline values, e.g., CD14+ cells routinely had a higher percentage of cells with Ca²⁺ᵢ levels above the threshold even under “resting” conditions. Results using serotonin as a ligand control are shown at B3 and B5. The compound listed as D10 activated the CD4+ population selectively, while the compound listed as 17 activated the CD14+ population selectively. The compound listed as N8 activated all three cell populations. This illustrates how an automated system can be used to run multiplexed analyses of primary tissue cell subpopulations to detect cell type selective activities to a series of test compounds.

Aspects of the present invention have been illustrated and described in detail with reference to particular embodiments by way of example only, and not by way of limitation. It will be appreciated that various modifications and alterations may be made to the exemplary embodiments without departing from the scope and contemplation of the present disclosure. It is intended, therefore, that the invention be considered as limited only by the scope of the appended claims.

1. A multiplexed screening method comprising:
(a) developing a plurality of cell populations to be screened, wherein each cell population expresses a ligand target;
(b) color-coding each of the plurality of cell populations by staining at least one cell population with a fluorochrome, to yield a distinct optical signature for each color-coded cell population, and loading each cell population with a fluorescent indicator dye to monitor a cellular response;
(c) combining the color-coded cell populations to form a mixed cell suspension;
(d) contacting the mixed cell suspension with a ligand or control compound;
(e) analyzing the mixed cell suspension by a single cell analysis system, comprising using one or more light sources to excite each color-coded cell in the mixed cell suspension and collecting fluorescence emissions from each excited cell to measure the distinct optical signature and the cellular response of each cell; and
(f) resolving each of the plurality of cell populations in the mixed cell suspension by deconvoluting data collected in step (e).

2. The method of claim 1, wherein the cell populations are stained with at least one fluorochrome selected from FM 1-43, FM 4-64, DiO, DiI, DiA, DiD, DiR, PKH 2, PKH26, Bodipy 665, LysoSensor Blue, Hoescht 33232, fluorescein, coumarin, rhodamine. Alexa 488, Alexa 500, Alexa 514, phycoerythrin, Alexa 594, Alexa 647, Alexa 660 and Alexa 750.

3. The method of claim 1, wherein the cell populations are directly stained with at least one fluorochrome.

4. The method of claim 1, wherein the cell populations are first labelled with an anchor molecule and the at least one fluorochrome binds to the anchor molecule.

5. The method of claim 4, wherein the anchor molecule is a biotinylated molecule and the fluorochrome is conjugated to a biotin-binding molecule.

6. The method of claim 5, wherein the fluorochrome is conjugated to streptavidin or avidin.

7. The method of claim 4, wherein the anchor molecule is an avidin- or streptavidin-conjugated molecule and the fluorochrome is conjugated to an avidin- or streptavidin-binding molecule.

8. The method of claim 7, wherein the fluorochrome is conjugated to biotin or a biotin derivative.

9. The method of claim 4, wherein the flurochrome is Alexa 488, Alexa 500, Alexa 514, phycoerythrin, Alexa 594, Alexa 647, Alexa 660 or Alexa 750.

10. The method of claim 1, wherein color-coding the plurality of cell populations in step (b) comprises not staining one of the plurality of cell populations, to yield a distinct optical signature for the unstained cell population.

11. The method of claim 1, wherein the cellular response is a cellular response to the ligand.

12. The method of claim 11, wherein the cellular response to the ligand is internal Ca²⁺ mobilization (Ca²⁺ₒ).

13. The method of claim 12, wherein the indicator is Indo-1, Fluo-3, Fluo-4, Oregon Green 488 BAPTA, Calcium Green, X-rhod-1 or Fura Red.

14. The method of claim 11, wherein the cellular response to a ligand is a change in membrane potential.

15. The method of claim 1, wherein the mixed cell suspension is incubated with the ligand for between about 0.1 second to about 1 week before analyzing.

16. The method of claim 15, further comprising adding additional indicator dyes before analyzing.

17. The method of claim 15, wherein the mixed cell suspension is incubated with the ligand for between about 1 second to about 5 seconds before analyzing.

18. The method of claim 15, wherein the mixed cell suspension is incubated with the ligand for between about 1 minute to about 1 hour.

19. The method of claim 15, wherein the mixed cell suspension is incubated with the ligand for between about 1 hour to about 48 hours before analyzing.

20. The method of claim 1, wherein the plurality of cell populations comprises a cell population expressing an endogenous ligand target.

21. The method of claim 1, wherein the plurality of cell populations comprises a cell population expressing a transfected ligand target.

22. The method of claim 1, wherein the plurality of cell populations comprises a cell population expressing a regulatable ligand target.

23. The method of claim 1, wherein the plurality of cell populations comprises a cell population expressing a ligand target with an expression tag.

24. The method of claim 1, wherein the plurality of cell populations comprises a cell population expressing a wild type ligand target.

25. The method of claim 1, wherein the plurality of cell populations comprises a cell population expressing a variant ligand target.

26. The method of claim 25, wherein the variant ligand target is a naturally occurring variant ligand target.

27. The method of claim 25, wherein the variant ligand target is a mutant ligand target.

28. The method of claim 1, wherein the plurality of cell populations comprises a cell population expressing a wild type ligand target and a cell population expressing a variant ligand target.

29. The method of claim 1, comprising a plurality of cell populations expressing a plurality of variant ligand targets.

30. The method of claim 29, wherein analyzing the mixed cell suspension and resolving each of the plurality of cell populations in the mixed cell suspension identifies cell populations having increased response to the ligand.

31. The method of claim 30, wherein cells having increased response to the ligand are isolated.

32. The method of claim 1, wherein the single cell analysis system is a flow cytometry system.

33. The method of claim 32, wherein the flow cytometry system further comprises fluorescence activated cell sorting (FACS).

34. The method of claim 33, wherein cells are sorted using FACS.

35. The method of claim 1, wherein the single cell analysis system comprises a liquid handling apparatus operative to prepare a mixed cell suspension, a sample analysis apparatus, and an injection guide coupled to the analysis apparatus, wherein the injection guide is operative to receive the mixed cell suspension from the liquid handling apparatus and provide the mixed cell suspension to a fluidic system of the sample analysis apparatus.
